(19) United States
(12) Patent Application Publication
(10) Pub. No.: US 2006/0217362 A1
(43) Pub. Date: Sep. 28, 2006

(54) Novel compounds useful for bradykinin B1 receptor antagonism

(76) Inventors: Jay S. Tung, Belmont, CA (US); Darren D. Dressen, San Mateo, CA (US); R. Jeffrey Neitz, San Francisco, CA (US); Michael A. Pleiss, Sunnyvale, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/312,757

(22) Filed: Dec. 21, 2005

Related U.S. Application Data
(60) Provisional application No. 60/640,021, filed on Dec. 29, 2004.

(51) Int. Cl. A61K 31/5513; A61K 31/495; A61K 31/496; A61K 31/4545; A61K 31/445; A61K 31/397

(52) U.S. Cl. 514/210.01; 514/221; 514/357; 514/602; 514/255.02; 514/317; 514/316; 514/252.14

(57) ABSTRACT

Disclosed are compounds that are bradykinin B₁ receptor antagonists and are useful for treating diseases, or relieving adverse symptoms associated with disease conditions, in mammals mediated by bradykinin B₁ receptor. Certain of the compounds exhibit increased potency and are also expected to exhibit increased duration of action.

### CROSS-REFERENCE TO RELATED APPLICATION

This application claims the benefit of priority under 35 U.S.C. § 119(e) to U.S. Provisional Application 60/640,021, filed Dec. 29, 2004.

### FIELD OF THE PRESENT INVENTION

The present invention is directed to compounds and methods useful as bradykinin B₁ receptor antagonists which may relieve adverse symptoms in mammals mediated, at least in part, by a bradykinin B₁ receptor including pain, inflammation, septic shock, scarring processes, and the like.

### BACKGROUND OF THE PRESENT INVENTION

Bradykinin (“BK”) or kinin-9 is a vasoactive nine-amino acid peptide (Arg-Pro-Pro-Gly-Phe-Ser-Pro-Phe-Arg) that is formed locally in body fluids and tissues from the plasma precursor kininogen during inflammatory processes. It is a potent but short-lived agent of arteriolar dilation and increased capillary permeability. BK is also known to be one of the most potent naturally occurring stimulator of C-fiber afferents mediating pain, and a physiologically active component of the kallikrein-kinin system.

BK, the nonapeptide sequence pH-Arg¹-Pro²-Pro³-Gly⁴-Phe⁵-Ser⁶-Pro⁷-Phe⁸-Arg⁹-OH (“SEQ. ID. NO. 1”) is formed by the action of plasma kallikrein, which hydrolyses the sequence out of the plasma globulin kininogen. Plasma kallikrein circulates as an inactive zymogen, from which active kallikrein is released by Hageman factor. Glandular kallikrein cleaves kininogen one residue earlier to give the decapeptide Lys-bradykinin (kallidin, Lys-BK) (“SEQ. ID. NO. 2”). Met-Lys-bradykinin (“SEQ. ID. NO. 3”) is also formed, perhaps by the action of leukocyte kallikrein. Pharmacologically important analogues include des-Arg⁹ (amino acid 1-8 of SEQ. ID. NO. 1) or BK₁₋₈ and Ile-Ser-bradykinin (or T-kinin) (“SEQ. ID. NO. 4”), [Hyp³]bradykinin (“SEQ. ID. NO. 5”), and [Hyp⁴]bradykinin (“SEQ. ID. NO. 6”). See e.g., Oxford Dictionary of Biochemistry and Molecular Biology, Oxford University Press (2001). BK is also released from mast cells during asthma attacks, from gut walls as a gastrointestinal vasodilator, from damaged tissues as a pain signal, and may be a neurotransmitter.

BK is also a powerful blood-vessel dilator, increasing vascular permeability and causing a fall in blood pressure, an edema-producing agent, and a stimulator of various vascular and non-vascular smooth muscles in tissues such as uterus, gut and bronchiole. BK is formed in a variety of inflammatory conditions and in experimental anaphylactic shock. The kinin/kininogen activation pathway has also been described as playing a pivotal role in a variety of physiologic and pathophysiologic processes, being one of the first systems to be activated in the inflammatory response and one of the most potent simulators of: (i) phospholipase A₂ and, hence, the generation of prostaglandins and leukotrienes; and (ii) phospholipase C and thus, the release of inositol phosphates and diacylgylcerol. These effects are mediated predominantly via activation of BK receptors of the BK₂ type.

A BK receptor is any membrane protein that binds BK and mediates its intracellular effects. Two recognized types of receptors are B₁ and B₂. On B₁ the order of potency is,
des-Arg⁹-bradykinin (BK₁₋₈ or amino acid 1-8 of SEQ. ID. NO. 1)=kallidin (SEQ. ID. NO. 2)>BK (SEQ. ID. NO. 1).
   On B₂ the order of potency is,
kallidin (SEQ. ID. NO. 2)>BK (SEQ. ID. NO. 1)>>BK₁₋₈.
   Hence, BK₁₋₈ is a powerful discriminator. See e.g., Oxford Dictionary of Biochemistry and Molecular Biology, Oxford University Press (2001).

B₁ receptors are considerably less common than B₂ receptors, which are present in most tissues. The rat B₂ receptor is a seven-transmembrane-domain protein that has been shown on activation to stimulate phosphoinositide turnover. Inflammatory processes induce the B1 subtype. See, e.g., Marceau, Kinin B₁ Receptors: A Review, Immunopharmacology, 30:1-26 (1995) (incorporated herein by reference in full). The distribution of receptor B₁ is very limited since this receptor is only expressed during states of inflammation. BK receptors have been cloned for different species, notably the human B1 receptor (See e.g., J. G. Menke, et al., J. Biol. Chem., 269(34):21583-21586 (1994) (incorporated herein by reference in full) and J. F. Hess, Biochem. Human B₂ Receptor, Biophys. Res. Commun., 184:260-268 (1992) (incorporated herein by reference in full)). For examples, there is: B₁, database code BRB1_HUMAN, 353 amino acids (40.00 kDa); and B₂, database code BRB2_HUMAN, 364 amino acids (41.44 kDa). See, e.g., Oxford Dictionary of Biochemistry and Molecular Biology, Oxford University Press (2001).

Two major kinin precursor proteins, high molecular weight and low molecular weight kininogen, are synthesized in the liver, circulate in plasma, and are found in secretions such as urine and nasal fluid. High molecular weight kininogen is cleaved by plasma kallikrein, yielding BK, or by tissue kallikrein, yielding kallidin. Low molecular weight kininogen, however, is a substrate only for tissue kallikrein. In addition, some conversion of kallidin to BK may occur inasmuch as the amino terminal lysine residue of kallidin is removed by plasma aminopeptidases. Plasma half-lives for kinins are approximately 15 seconds, with a single passage through the pulmonary vascular bed resulting in 80-90% destruction. The principle catabolic enzyme in vascular beds is the dipeptidyl carboxypeptidase kininase II or angiotensin-converting enzyme (ACE). A slower acting enzyme, kininase I, or carboxypeptidase N, which removes the carboxyl terminal Arg, circulates in plasma in great abundance. This suggests that it may be the more important catabolic enzyme physiologically. Des-Arg⁹-bradykinin (amino acid 1-8 of SEQ. ID. NO. 1) as well as des-Arg¹⁰-kallidin (amino acid 1-9 of SEQ. ID. NO. 2) formed by kininase I acting on BK or kallidin, respectively, are acting BK₁ receptor agonists, but are relatively inactive at the more abundant BK₂ receptor at which both BK and kallidin are potent agonists.

Direct application of BK to denuded skin or intra-arterial or visceral injection results in the sensation of pain in mammals including humans. Kinin-like materials have been isolated from inflammatory sites produced by a variety of stimuli. In addition, BK receptors have been localized to nociceptive peripheral nerve pathways and BK has been demonstrated to stimulate central fibers mediating pain sensation. BK has also been shown to be capable of causing hyperalgesia in animal models of pain. (See, e.g., R. M. Burch, et al., Bradykinin Receptor Antagonists, Med. Res. Rev., 10(2):237-269 (1990) (incorporated herein by reference in full); Clark, W. G. Kinins and the Peripheral Central Nervous Systems, Handbook of Experimental Pharmacology, Vol. XXV: Bradykinin, Kallidin, and Kallikrein. Erdo, E. G. (Ed.), 311-322 (1979) (incorporated herein by reference in full)).

These observations have led to considerable attention being focused on the use of BK antagonists as analgesics. A number of studies have demonstrated that BK antagonists are capable of blocking or ameliorating both pain as well as hyperalgesia in mammals including humans. See, e.g., Ammons, W. S., et al., Effects of Intracardiac Bradykinin on T₂-T₅ Medial Spinothalamic Cells, American Journal of Physiology, 249, R145-152 (1985) (incorporated herein by reference in full); Clark, W. G. Kinins and the Peripheral Central Nervous Systems, Handbook of Experimental Pharmacology, Vol. XXV: Bradykinin, Kallidin, and Kallikrein. Erdo, E. G. (Ed.), 311-322 (1979) (incorporated herein by reference in full)); Costello, A. H. et al., Suppression of Carageenan-Induced Hyperalgesia, Hyperthermia and Edema by a Bradykinin Antagonist, European Journal of Pharmacology, 171:259-263 (1989) (incorporated herein by reference in full); Laneuville, et al., Bradykinin Analogue Blocks Bradykinin-induced Inhibition of a Spinal Nociceptive Reflex in the Rat, European Journal of Pharmacology, 137:281-285 (1987) (incorporated herein by reference in full); Steranka, et al., Antinociceptive Effects of Bradykinin Antagonists, European Journal of Pharmacology, 136:261-262 (1987) (incorporated herein by reference in full); and Steranka, et al., Bradykinin as a Pain Mediator: Receptors are Localized to Sensory Neurons, and Antagonists have Analgesic Actions, Neurobiology, 85:3245-3249 (1987) (incorporated herein by reference in full).

Currently accepted therapeutic approaches to analgesia have significant limitations. While mild to moderate pain can be alleviated with the use of non-steroidal anti-inflammatory drugs and other mild analgesics, severe pain, such as that accompanying surgical procedure, burns and severe trauma requires the use of narcotic analgesics. These drugs carry the limitations of abuse potential, physical and psychological dependence, altered mental status and respiratory depression, which significantly limit their usefulness.

Prior efforts in the field of BK antagonists indicate that such antagonists can be useful in a variety of roles. These include use in the treatment of burns, perioperative pain, migraine and other forms of pain, shock, central nervous system injury, asthma, rhinitis, premature labor, inflammatory arthritis, inflammatory bowel disease, neuropathic pain, etc. For example, Whalley, et al., has demonstrated that BK antagonists are capable of blocking BK-induced pain in a human blister base model. See Whalley, et al., in Naunyn Schmiederberg's Arch. Pharmacol., 336:652-655 (1987) (incorporated herein by reference in full). This suggests that topical application of such antagonists would be capable of inhibiting pain in burned skin, e.g., in severely burned patients that require large doses of narcotics over long periods of time and for the local treatment of relatively minor burns or other forms of local skin injury.

The management of perioperative pain requires the use of adequate doses of narcotic analgesics to alleviate pain while not inducing excessive respiratory depression. Post-operative narcotic-induced hypoventilation predisposes patients to collapse of segments of the lungs (a common cause of post-operative fever), and frequently delays discontinuation of mechanical ventilation. The availability of a potent non-narcotic parenteral analgesic could be a significant addition to the treatment of perioperative pain. While no currently available BK antagonist has the appropriate pharmacodynamic profile to be used for the management of chronic pain, anesthesiologists and surgeons in the management of perioperative pain already commonly use frequent dosing and continuous infusions.

Several lines of evidence suggest that the kallikrein/kinin pathway may be involved in the initiation or amplification of vascular reactivity and sterile inflammation in migraine. (See, e.g., Back, et al., Determination of Components of the Kallikrein-Kinin System in the Cerebrospinal Fluid of Patients with Various Diseases, Res. Clin. Stud. Headaches, 3:219-226 (1972) (incorporated herein by reference in full). Because of the limited success of both prophylactic and non-narcotic therapeutic regimens for migraine, as well as the potential for narcotic dependence in these patients, the use of BK antagonists offers a highly desirable alternative approach to the therapy of migraine.

BK is produced during tissue injury and can be found in coronary sinus blood after experimental occlusion of the coronary arteries. In addition, when directly injected into the peritoneal cavity, BK produces a visceral type of pain. (See, e.g., Ness, et al., Visceral pain: a Review of Experimental Studies, Pain, 41:167-234 (1990) (incorporated herein by reference in full). While multiple other mediators are also clearly involved in the production of pain and hyperalgesia in settings other than those described above, it is also believed that antagonists of BK have a place in the alleviation of such forms of pain as well.

Shock related to bacterial infections is a major health problem. It is estimated that 400,000 cases of bacterial sepsis occur in the United States yearly; of those, 200,000 progress to shock, and 50% of these patients die. Current therapy is supportive, with some suggestion in recent studies that monoclonal antibodies to Gram-negative endotoxin may have a positive effect on disease outcome. Mortality is still high, even in the face of this specific therapy, and a significant percentage of patients with sepsis are infected with Gram-positive organisms that would not be amenable to anti-endotoxin therapy.

Multiple studies have suggested a role for the kallikrein/kinin system in the production of shock associated with endotoxin. See, e.g., Aasen, et al., Plasma kallikrein Activity and Prekallikrein Levels during Endotoxin Shock in Dogs, Eur. Surg., 10:5062 (1977) (incorporated herein by reference in full); Aasen, et al., Plasma Kallikrein-Kinin System in Septicemia, Arch. Surg., 118:343-346 (1983) (incorporated herein by reference in full); Katori, et al., Evidence for the Involvement of a Plasma Kallikrein/Kinin System in the Immediate Hypotension Produced by Endotoxin in Anaesthetized Rats, Br. J. Pharmacol., 98:1383-1391 (1989) (incorporated herein by reference in full); Marceau, et al., Pharmacology of Kinins: Their Relevance to Tissue Injury and Inflammation, Gen. Pharmacol., 14:209-229 (1982) (incorporated herein by reference in full). Recent studies using newly available BK antagonists have demonstrated in animal models that these compounds can profoundly affect the progress of endotoxic shock. See, e.g., Weipert, et al., Brit J. Pharm., 94:282-284 (1988) (incorporated herein by reference in full). Less data is available regarding the role of BK and other mediators in the production of septic shock due to Gram-positive organisms. However, it appears likely that similar mechanisms are involved. Shock secondary to trauma, while frequently due to blood loss, is also accompanied by activation of the kallikrein/kinin system. See, e.g., Haberland, The Role of Kininogenases, Kinin Formation and Kininogenase Inhibitor in Post Traumatic Shock and Related Conditions, Klinische Woochen-Schrift, 56:325-331 (1978) (incorporated herein by reference in full).

Numerous studies have also demonstrated significant levels of activity of the kallikrein/kinin system in the brain. Both kallikrein and BK dilate cerebral vessels in animal models of CNS injury. See, e.g., Ellis, et al., Inhibition of Bradykinin- and Kallikrein-Induced Cerebral Arteriolar Dilation by Specific Bradykinin Antagonist, Stroke, 18:792-795 (1987) (incorporated herein by reference in full); and Kamitani, et al., Evidence for a Possible Role of the Brain Kallikrein-Kinin System in the Modulation of the Cerebral Circulation, Circ. Res., 57:545-552 (1985) (incorporated herein by reference in full). BK antagonists have also been shown to reduce cerebral edema in animals after brain trauma. Based on the above, it is believed that BK antagonists should be useful in the management of stroke and head trauma.

Other studies have demonstrated that BK receptors are present in the lung, that BK can cause bronchoconstriction in both animals and man, and that a heightened sensitivity to the bronchoconstrictive effect of BK is present in asthmatics. Some studies have been able to demonstrate inhibition of both BK and allergen-induced bronchoconstriction in animal models using BK antagonists. These studies indicate a potential role for the use of BK antagonists as clinical agents in the treatment of asthma. See, e.g., Barnes, Inflammatory Mediator Receptors and Asthma, Am. Rev. Respir. Dis., 135:S26-S31 (1987) (incorporated herein by reference in full); R. M. Burch, et al., Bradykinin Receptor Antagonists, Med. Res. Rev., 10(2):237-269 (1990) (incorporated herein by reference in full); Fuller, et al., Bradykinin-induced Bronchoconstriction in Humans, Am. Rev. Respir. Dis., 135:176-180 (1987) (incorporated herein by reference in full); Jin, et al., Inhibition of Bradykinin-Induced Bronchoconstriction in the Guinea-Pig by a Synthetic B₂ Receptor Antagonist, Br. J. Pharmacol., 97:598-602 (1989) (incorporated herein by reference in full), and Polosa, et al., Contribution of Histamine and Prostanoids to Bronchoconstriction Provoked by Inhaled Bradykinin in Atopic Asthma, Allergy, 45:174-182 (1990) (incorporated herein by reference in full). BK has also been implicated in the production of histamine and prostanoids to bronchoconstriction provoked by inhaled BK in atopic asthma. See, e.g., Polosa, et al., Contribution of Histamine and Prostanoids to Bronchoconstriction Provoked by Inhaled Bradykinin in Atopic Asthma, Allergy, 45:174-182 (1990) (incorporated herein by reference in full). BK has also been implicated in the production of symptoms in both allergic and viral rhinitis. These studies include the demonstration of both kallikrein and BK in nasal lavage fluids and that levels of these substances correlate well with symptoms of rhinitis. See, e.g., Baumgarten, et al., Concentrations of Glandular Kallikrein in Human Nasal Secretions Increase During Experimentally Induced Allergic Rhinitis, J. Immunology, 137:1323-1328 (1986) (incorporated herein by reference in full); Jin, et al., Inhibition of Bradykinin-Induced Bronchoconstriction in the Guinea-Pig by a Synthetic B₂ Receptor Antagonist, Br. J. Pharmacol., 97:598-602 (1989), and Proud, et al., Nasal Provocation with Bradykinin Induces Symptoms of Rhinitis and a Sore Throat, Am. Rev. Respir Dis., 137:613-616 (1988) (incorporated herein by reference in full).

In addition, studies have demonstrated that BK itself can cause symptoms of rhinitis. Steward et. al, discusses peptide BK antagonists and their possible use against effects of BK. See, e.g., Steward and Vavrek in Chemistry of Peptide Bradykinin Antagonists Basic and Chemical Research, R. M. Burch (Ed.), pages 51-96 (1991) (incorporated herein by reference in full).

A great deal of research effort has been expended towards developing such antagonists with improved properties. However, notwithstanding extensive efforts to find such improved BK antagonists, there remains a need for additional and more effective BK antagonists. Two of the major problems with presently available BK antagonists are their low levels of potency and their extremely short durations of activity. Thus, there is a special need for BK antagonists having increased potency and for duration of action.

Two generations of peptidic antagonists of the B2 receptor have been developed. The second generation has compounds two orders of magnitude more potent as analgesics than first generation compounds. The most important derivative was icatibant. The first non-peptidic antagonist of the B2 receptor, described in 1993, has two phosphonium cations separated by a modified amino acid. Many derivatives of this di-cationic compound have been prepared. Another non-peptidic compound antagonist of B2 is the natural product Martinelline. See, e.g., Elguero, et al., Nonconventional Analgesics: Bradykinin Antagonists, An. R. Acad. Farm., 63(1):173-90 (Spa) (1997) (incorporated herein by reference in full); and Seabrook, et al., Expression of B1 and B2 Bradykinin Receptor mRNA and Their Functional Roles in Sympathetic Ganglia and Sensory Dorsal Root Ganglia Neurons from Wild-type and B2 Receptor Knockout Mice, Neuropharmacology, 36(7):1009-17 (1997) (incorporated herein by reference in full).

U.S. Pat. No. 3,654,275 teaches that certain 1,2,3,4-tetrahydro-1-acyl-3-oxo-2-quinoxalinecarboxamides have anti-inflammatory activity. See, e.g., McManus, U.S. Pat. No. 3,654,275, Quinoxalinecarboxamide Antiinflammatory Agents, issued Apr. 4, 1972 (incorporated herein by reference in full).

International Patent Application WO 03/007958 filed on Jul. 2, 2002 and published on Jan. 30, 2003 discloses tetrahydroquinoxalines acting as BK antagonists. See, e.g., Beyreuther, B.; et al., International Patent Application WO 03/007958 A1 published on Jan. 30, 2003 (incorporated herein by reference in full).

U.S. Pat. No. 5,916,908 teaches the use of 3,5-disubstituted pyrazoles or 3,4,5-trisubstituted pyrazoles as kinase inhibitors. See, e.g., Giese, et al., U.S. Pat. No. 5,916,908, issued Jun. 29, 1999 (incorporated herein by reference in full).

Japanese Patent Application Serial No. 49100080 teaches 2-aminopyrazoles as anti-inflammatory agents. See, e.g., Yoshida, et al., Japanese Patent Application Serial No. 49100080 (incorporated herein by reference in full).

Currently there is no marketed therapeutic agents for the inhibition of bradykinin B₁ receptor. In view of the above, compounds which are bradykinin B₁ receptor antagonists would be particularly advantageous in treating those diseases mediated by bradykinin B₁ receptor.

Accordingly, it is an object of the present invention to provide compounds and methods of treatment useful as therapeutic agent for the inhibition of bradykinin B₁ receptor. It is also an object of the present invention to provide compounds and methods of treatment useful in treating diseases, disorders, and conditions, which benefit from inhibition of the bradykinin B₁ receptor.

The present invention accomplishes these objectives and provides further related advantages.

### BRIEF SUMMARY OF THE PRESENT INVENTION

The present invention is directed to methods and compounds useful in treating diseases, disorders, and conditions, which benefit from inhibition of the bradykinin B₁ receptor.

In an embodiment, the present invention provides a method of preventing or treating conditions which benefit from inhibition of the bradykinin B₁ receptor, comprising:

administering to a host in need thereof a composition comprising a therapeutically effective amount of at least one compound of formula (I), 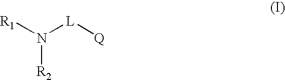
or pharmaceutically acceptable salts thereof, wherein R₁, R₂, L and Q are as defined below.

In another embodiment, the present invention provides an article of manufacture, comprising (a) at least one dosage form of at least one compound of formula (I), 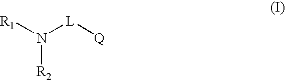
or pharmaceutically acceptable salt thereof, wherein R₁, R₂, L and Q are defined below, (b) a package insert providing that a dosage form comprising a compound of formula (I) should be administered to a patient in need of therapy for disorders, conditions or diseases which benefit from inhibition of the bradykinin B₁ receptor, and (c) at least one container in which at least one dosage form of at least one compound of formula (I) is stored.

In another embodiment, the present invention provides a packaged pharmaceutical composition for treating diseases, disorders, and conditions, which benefit from inhibition of the bradykinin B₁ receptor, (a) a container which holds an effective amount of at least one compound of formula (I), or a pharmaceutically acceptable salt thereof, wherein R₁, R₂, L and Q are as defined below, and (b) instructions for using the pharmaceutical composition.

BK is a kinin that plays an important role in the patho-physiological processes accompanying acute and chronic pain and inflammation. BKs, like other related kinins, are autocoid peptides produced by the catalytic action of kallikrein enzymes on plasma and tissue precursors termed kininogens. Inhibition of bradykinin B1 receptors by compounds that are bradykinin B1 antagonists or inverse agonists would provide relief from maladies that mediate undesirable symptoms through a bradykinin B1 receptor pathway.

The compounds of this invention are bradykinin B₁ receptor antagonists and therefore are suitable for use in blocking or ameliorating pain as well as hyperalgesia in mammals. Such compounds would be effective in the treatment or prevention of pain including, for example, bone and joint pain (osteoarthritis), repetitive motion pain, dental pain, pain associated with cancer, myofascial pain (muscular injury, fibromyalgia), perioperative pain (general surgery, gynecological) and chronic pain. In particular, inflammatory pain such as, for example, inflammatory airways disease (chronic obstructive pulmonary disease) would be effectively treated by bradykinin B1 antagonist compounds.

The compounds of this invention are also useful in the treatment of disease conditions in a mammal that are mediated, at least in part, by a bradykinin B₁ receptor. Examples of such disease conditions include asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture. They may be used subsequent to surgical intervention (e.g. as post-operative analgesics) and to treat inflammatory pain of varied origins (e.g. osteoarthritis, rheumatoid arthritis, rheumatic disease, tenosynovitis and gout), as well as for the treatment of pain associated with angina, menstruation, or cancer. They may also be used to treat diabetic vasculopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus or in the therapy of Crohn's disease, ulcerative colitis or pancreatitis. Such compounds may also be used therapeutically to treat hyperreactive airways and to treat inflammatory events associated with diseases or conditions affecting the airways (e.g., asthma), and to control, restrict or reverse airways hyperreactivity in asthma. They may be used to treat intrinsic and extrinsic asthma, including allergic asthma (atopic or non-atopic), as well as exercise-induced asthma, occupational asthma, asthma post-bacterial infection, other non-allergic asthmas and “wheezy-infant syndrome”. They may also be effective against pneumoconiosis, including aluminosis, anthracosis, asbestosis, chalicosis, ptilosis, siderosis, silicosis, tabacosis and byssinosis, as well as adult respiratory distress syndrome, chronic obstructive pulmonary or diseases or conditions affecting the airways, bronchitis, allergic rhinitis, and vasomotor rhinitis. Additionally, they may be effective against liver disease, multiple sclerosis, atherosclerosis, Alzheimer's disease, septic shock (e.g., as anti-hypovolemic and/or anti-hypotensive agents), cerebral edema, headache, migraine, closed head trauma, irritable bowel syndrome and nephritis. Finally, such compounds are also useful as research tools (in vivo and in vitro).

As noted above, the compounds of this invention are typically administered to the mammal in the form of a pharmaceutical composition. Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985).

To enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 (each of which is incorporated herein by reference in full).

The amount administered to the patient will vary depending upon what is being administered, the purpose of the administration, such as prophylaxis or therapy, the state of the patient, the manner of administration, and the like all of which are within the skill of the attending clinician. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the inflammation, the age, weight and general condition of the patient, and the like.

The compositions administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 20 Fg to about 500 Fg per kilogram body weight, preferably about 100 Fg to about 300 Fg per kilogram body weight. Suitable dosage ranges for intranasal administration are generally about 0.1 pg to 1 mg per kilogram body weight. Effective doses can be extrapolated from dose-response curves derived from in vitro or animal model test systems.

### SUMMARY OF THE INVENTION

This invention is directed, in part, to compounds that are bradykinin B₁ receptor antagonist. It is also directed to compounds that are useful for treating diseases or relieving adverse symptoms associated with disease conditions in mammals, where the disease is mediated at least in part by bradykinin B₁ receptor. For example, inhibition of the bradykinin B₁ receptor is useful for the moderation of pain, inflammation, septic shock, the scarring process, etc. These compounds are preferably selective for antagonism of the B₁ receptor over the B₂ receptor. This selectivity may be therapeutically beneficial due to the up-regulation of the B₁ receptor following tissue damage or inflammation. Certain of the compounds exhibit increased potency and are expected to also exhibit an increased duration of action.

In an embodiment, this invention provides compounds of formula (I) 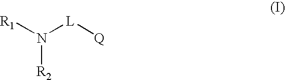
or pharmaceutically acceptable salts thereof, wherein
R₁ is selected from formulae (IIa), (IIb), (IIc), (IId), (IIe), (IIf), and (IIg);
(IIa) is 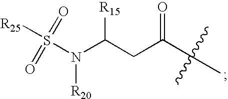
R₁₅, R₂₀, and R₂₅ are independently selected from hydrogen, alkyl, aryl, heteroaryl, alkylaryl, alkylheteroaryl, and —(CH₂)₀₋₆-T;
T is a monocyclic or bicyclic ring system of 5, 6, 7, 8, 9, 10, 11, or 12 atoms, wherein at least one bond in the monocyclic or bicyclic ring system is optionally a double bond, wherein the bicyclic ring system is optionally a fused or spiro ring system, wherein at least one ring in the monocyclic or bicyclic ring system is optionally aromatic, wherein at least one carbon atom in the monocyclic or bicyclic ring system is optionally replaced by a group independently selected from —O—, —C(O)—, —S(O)₀₋₂—, —C(═N—R₆)—, —N—, —NR₆—, —N((CO)₀₋₁R₂₆)—, and —N(SO₂R₂₆)—;
wherein R₁₅, R₂₀, and R₂₅ are independently optionally substituted with at least one R₂₆ group;
R₂₆ is selected from NO₂, CN, halogen, alkyl (optionally substituted with at least one halogen), alkoxy (optionally substituted with at least one halogen), alkylenedioxy (optionally substituted with at least one halogen), benzyloxy, phenyl, —NH₂, —OH, —CF₃, alkylamino, dialkylamino, oxo, —C(O)R₂₇, —COOR₂₇, —C(O)NR₂₇R_{27′}, —NR₂₇C(O)R_{27′}, alkyl, aryl, heteroaryl, cycloalkyl and heterocycloalkyl, each of which is optionally substituted with at least one group independently selected from halogen, —NH₂, —OH, —CN, —CF₃, alkylamino, haloalkyl, oxo, alkoxy, alkoxyalkyl, benzyloxy, alkyl, dialkylamino, —C(O)R₂₇, —COOR₂₇, —C(O)NR₂₇R_{27′}, and NR₂₇C(O)R_{27′};
R₂₇ and R_{27′} are independently selected from H, alkyl, aryl and heteroaryl, each of which is optionally substituted with at least one group independently selected from alkyl, halogen, alkoxy, OH, amino, monoalkylamino, dialkylamino, and CF₃;
(IIb) is 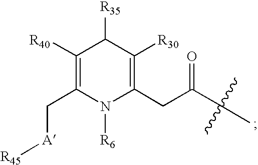
R₃₀ and R₄₀ are independently selected from H, —CO₂H and —CO₂alkyl;
R₃₅ is phenyl optionally substituted with at least one halogen;
A′ is selected from —(CH₂)₀₋₂—, —C(O)—, and —S(O)₀₋₂—;
R₄₅ is selected from azabicycloalkyl, azatricycloalkyl, bicycloalkyl, tricycloalkyl, and phenyl substituted at the 2-position with a group selected from
(a) alkyl optionally substituted with at least one group independently selected from amino, amino-alkoxy, phenylthio, alkyl-phenylthio, dialkylamino-alkoxy, alkylamino-alkoxy, alkylamino, di-alkylamino, hydroxy, alkoxy, piperazinyl, oxopyrrolidinyl, pyrrolidinyl, alkylenedioxy, acyloxy, oxo, morpholino, alkylaminocarbonyl-acylamino, alkoxycarbonyl-acylamino, alkoxycarbonylpiperazinyl, acylpiperazinyl, alkylthio, heterocyclic-alkoxy, (dialkylamino)(cycloalkyl)alkoxy, (alkylamino)(cycloalkyl)alkoxy, (amino)(cycloalkyl)alkoxy, phenylthio, and acylamino;
(b) alkoxy or alkylthio, wherein the alkoxy or alkylthio is optionally substituted with at least one group independently selected from amino, amino-alkoxy, phenylthio, alkyl-phenylthio, di-alkylamino-alkoxy, alkylamino-alkoxy, alkylamino, dialkylamino, hydroxy, alkoxy, piperazinyl, oxopyrrolidinyl, pyrrolidinyl, alkylenedioxy, acyloxy, oxo, morpholino, alkylaminocarbonyl-acylmino, alkoxycarbonyl-acylamino, alkoxycarbonylpiperazinyl, acylpiperazinyl, alkylthio, heterocyclic-alkoxy, (dialkylamino)(cycloalkyl)alkoxy, (alkylamino)(cycloalkyl)alkoxy and (amino)(cycloalkyl)alkoxy;
(c) amino, alkylamino, acylamino, aminoacetylamino, alkylsulfonylamino, halosubstituted-alkylsulfonylamino, halosubstituted-alkylamino and alkoxycarbonylaminoacetylamino;
(d) piperazinylcarbonyl, morpholinocarbony, nitro, cyano, hydroxy, alkylsulfonyl, alkylsulfinyl and di-alkylaminosulphenyl;
(e) alkylthio, acylthio, amino-acylthio, alkylsulfonylthio, halosubstituted-alkylthio and alkoxyaminoacetylthio; and
(f) azacycloalkyl optionally substituted with at least one group independently selected from oxo and alkyl;
(IIc) is 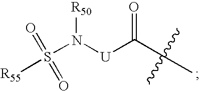
U is selected from alkyl and alkyl-O-alkyl;
R₅₀ is selected from hydrogen and alkyl optionally substituted with at least one group independently selected from halogen, amide, and phenyl;
R₅₅ is selected from hydrogen, alkyl, aryl, alkylaryl, and —(CH₂)₀₋₆-T;
wherein R₅₅ is optionally substituted with at least one R₂₆ group;
(IId) is 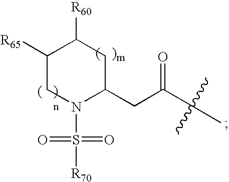
m is 0, 1, or 2;
n is 0, 1, 2, or 3;
R₆₀ and R₆₅ are independently selected from H and alkyl; or
R₆₀ and R₆₅ together form an aryl or heteroaryl ring optionally substituted with at least one group independently selected from halogen, —NH₂, —OH, —CN, —CF₃, alkylamino, oxo, alkoxy, dialkylamino, —C(O)R₂₇, —COOR₂₇, —C(O)NR₂₇R_{27′}, —NR₂₇C(O)R_{27′}, alkyl, aryl, heteroaryl, cycloalkyl and heterocyclyl, each of which is optionally substituted with at least one group independently selected from halogen, —NH₂, —OH, —CN, —CF₃, -alkylamino, haloalkyl, oxo, alkoxy, alkoxyalkyl, alkyl, dialkylamino, —C(O)R₂₇, —COOR₂₇, —C(O)NR₂₇R_{27′}, and NR₂₇C(O)R_{27′};
R₇₀ is selected from hydrogen, alkyl, aryl, alkylaryl, and (CH₂)₀₋₆-T; wherein R₇₀ is optionally substituted with at least one R₂₆ group;
(IIe) is 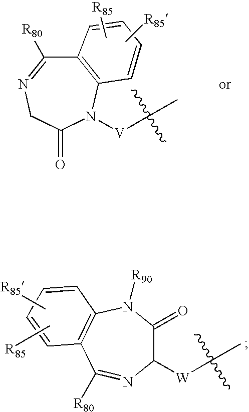
V is —(CH₂)₁₋₇—C(O)—;
W is selected from —NHC(O)—, —NHC(O)—(CH₂)₀₋₄aryl(CH₂)₀₋₄—, NHC(O)—(CH₂)₀₋₄aryl-C(O)—, —NHC(O)NH(CH₂)₀₋₄aryl(CH₂)₀₋₄—, —NHC(O)NH(CH₂)₀₋₄aryl-C(O)—, —OC(O)—, —OC(O)NH(CH₂)₀₋₄aryl(CH₂)₀₋₄—, —OC(O)NH(CH₂)₀₋₄aryl-C(O)—, —(CH₂)₁₋₃—C(O)—, —(CH₂)₁₋₃—C(O)—NH(CH₂)₀₋₄aryl(CH₂)₀₋₄—, and —(CH₂)₁₋₃—C(O)—NH(CH₂)₀₋₄aryl-C(O)—;
R₈₀ is selected from alkyl (optionally substituted with at least one halogen), alkyloxy-, alkyl, -alkyl-cycloalkyl, -alkylaryl, aryl, heteroaryl, and -alkylheteroaryl, wherein the aryl or heteroaryl is optionally substituted with at least one group independently selected from alkyl (optionally substituted with at least one halogen), alkyloxy, alkylcarboxy, alkylamido, OH, halogen, nitro, amino, and cyano; or
R₈₀ is selected from formulae (IIIa) and (IIIb), 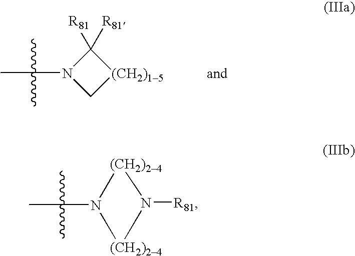
R₈₁ and R_{81′} are independently selected from H, alkyl (optionally substituted with at least one halogen), alkyloxy-, -alkylaryl, aryl, -alkylcycloalkyl, and cycloalkyl;
R₈₅ and R_{85′} are independently selected from H, NO₂, halogen, cyano, OH, amino, alkylthio-, alkyl (optionally substituted with at least one halogen), alkyloxy-, -alkylaryl, aryl, -alkylheteroaryl, heteroaryl, —C(O)—(CH₂)₀₋₂aryl, —C(O)—(CH₂)₀₋₂heteroaryl, —C(O)—O-aryl, —C(O)—O-heteroaryl, —C(O)—NH—(CH₂)₀₋₂aryl, —C(O)—N H(CH₂)₀₋₂heteroaryl, —C(O)—N(alkyl)-(CH₂)₀₋₂aryl, and —C(O)—N(alkyl)(CH₂)₀₋₂heteroaryl, wherein the aryl or heteroaryl is optionally substituted with at least one group independently selected from alkyl, alkyloxy-, alkylcarboxy-, alkylamido-, OH, halogen, nitro, amino, and cyano;
R₉₀ is selected from H, alkyl (optionally substituted with at least one halogen), alkyloxy-, -alkylaryl, -alkyl-cycloalkyl, and cycloalkyl;
(IIf) is 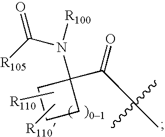
R₁₀₀ and R_{100′} are independently selected from H and alkyl;
R₁₀₅ is selected from alkyl (optionally substituted with at least one group independently selected from hydroxyethyl, halogen, nitro, cyano, —OR₁₁₁, —SR₁₁₁, —COR₁₁₁, —SO₂R₁₁₂, —CO₂R₁₁₁, —OC(O)R₁₁₁, —NR₁₁₃R₁₁₄, —NR₁₁₃C(O)R₁₁₁, —NR₁₁₃CO₂R₁₁₁, —C(O)NR₁₁₃R₁₁₄, and cycloalkyl), cycloalkyl (optionally substituted with at least one group independently selected from halogen, nitro, cyano and phenyl), (CH₂)₀₋₂-aryl (optionally substituted with at least one group independently selected from halogen, nitro, cyano, OR₁₁₁, SR₁₁₁, CO₂R₁₁₁, alkyl and haloalkyl), —(CH₂)₀₋₂-T (optionally substituted with at least one group independently selected from halogen, nitro, cyano, OR₁₁₁, SR₁₁₁, alkyl and haloalkyl), —CO₂R₁₁₁, and —C(O)NR₁₁₃R₁₁₄;
R₁₁₀ and R_{110′} are independently selected from hydrogen, halogen, and alkyl optionally substituted with at least one group independently selected from halogen, OR₁₁₁, OC(O)R₁₁₁, S(O)₀₋₂R₁₁₂, OS(O)₂R₁₁₂, and NR₁₀₀R_{100′}, or
R₁₁₀ and R_{110′} together with the carbon atom to which they are both attached form an exo-cyclic methylene optionally substituted with at least one group selected from alkyl (optionally substituted with at least one halogen) and alkyloxy;
R₁₁₁ is selected from hydrogen, alkyl (optionally substituted with at least one halogen), phenyl (optionally substituted with at least one group independently selected from halogen, cyano, nitro, OH, alkyloxy, cycloalkyl and alkyl optionally substituted with at least one halogen), cycloalkyl, and pyridyl optionally substituted with at least one group independently selected from halogen and alkyl;
R₁₁₂ is selected from alkyl (optionally substituted with at least one halogen), alkyloxy, and phenyl optionally substituted with at least one group independently selected from halogen, cyano, nitro, OH, alkyloxy, cycloalkyl and alkyl optionally substituted with at least one halogen;
R₁₁₃ and R₁₁₄ are independently selected from hydrogen, alkyl (optionally substituted with at least one group independently selected from halogen, amino, monoalkylamino, dialkylamino, and SO₂R₁₁₂), —(CH₂)₀₋₂-phenyl (optionally substituted with at least one group independently selected from halogen, cyano, nitro, OH, alkyloxy, cycloalkyl and alkyl (optionally substituted with at least one halogen)), and cycloalkyl; or
R₁₁₃ and R₁₁₄ together with the nitrogen atom to which they are attached form a 4-, 5-, or 6-membered ring wherein at least one carbon atom within the ring is optionally replaced with a group selected from —N—, —NR₂₆—, —S—, and —O—; or
R₁₁₃ and R₁₁₄ together with the nitrogen atom to which they are attached form a cyclic imide;
(IIg) is 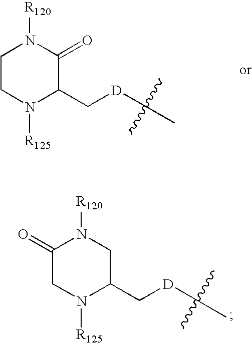
D is selected from —(CH₂)₀₋₆C(O)—, —(CH₂)₀₋₆NR₁₂₁C(O)—, —(CH₂)₀₋₆NR₁₂₁—, —(CH₂)₀₋₆O—, —C(O)—, —(CH₂)₀₋₆CO₂—, —(CH₂)₀₋₆S(O)₀₋₂—, —(CH₂)₀₋₆S—, —HC═CH—, and —(CH₂)₀₋₆—;
R₁₂₀ is selected from hydrogen, alkyl (optionally substituted with at least one halogen), -alkyl-aryl, —S(O)₀₋₂R₁₂₃′, cycloalkyl, —(CH₂)₀₋₆C(O)R₁₂₃, —(CH₂)₀₋₆CO₂R₁₂₃, and —(CH₂)₀₋₆C(O)NR₁₂₁R_{121′};
R₁₂₁ and R_{121′} are independently selected from hydrogen, alkyl (optionally substituted with at least one halogen), cycloalkyl, and aryl optionally substituted with at least one group independently selected from alkyl, halogen, nitro, cyano, OH, —O-alkyl (optionally substituted with at least one halogen), —C(O)OR₁₂₂, —C(O)NR₁₂₂R_{122′}, and —NR₁₂₂R_{122′}; or
R₁₂₁ and R_{121′} and the nitrogen atom to which they are attached together form a 4, 5, 6, or 7 membered ring, optionally; comprising a heteroatom selected from —O—, —S—, and —N(R₆)—;
R₁₂₂ is selected from hydrogen and alkyl;
R₁₂₃ is selected from hydrogen, alkyl (optionally substituted with at least one halogen), cycloalkyl, aryl (optionally substituted with at least one group independently selected from alkyl, halogen, nitro, cyano, —OH, O-alkyl (optionally substituted with at least one halogen), and NR₁₂₁R_{121′}), and heteroaryl (optionally substituted with at least one group independently selected from alkyl, halogen, nitro, cyano, —OH, O-alkyl (optionally substituted with at least one halogen)), and NR₁₂₁R_{121′};
R_{123′} is selected from alkyl (optionally substituted with at least one halogen), cycloalkyl, aryl (optionally substituted with at least one group independently selected from alkyl, halogen, nitro, cyano, —OH, O-alkyl (optionally substituted with at least one halogen), and NR₁₂₁R_{121′}), and heteroaryl (optionally substituted with at least one group independently selected from alkyl, halogen, nitro, cyano, OH, O-alkyl (optionally substituted with at least one halogen)), and NR₁₂₁R_{121′};
R₁₂₅ is selected from —(CH₂)₁₋₆CO₂R₁₂₃, —(CH₂)₁₋₆C(O)NR₁₂₁R_{121′}, —S(O)₀₋₂R₁₂₃, —C(O)R₁₂₃, —CO₂R₁₂₃, and alkyl (optionally substituted with at least one group independently selected from halogen, cyano, and aryl (optionally substituted with at least one group independently selected from halogen, cyano, —OR₁₂₃, —NR₁₂₁R_{121′}, —C(O)NR₁₂₁R_{121′}, and phenyl (optionally substituted with at least one group independently selected from —CO₂R₁₂₃, halogen, nitro, cyano, —OR₁₂₃, and NR₁₂₁R_{121′}));
R₂ is selected from hydrogen, heterocycloalkyl, heteroaryl, and aryl, wherein the heterocycloalky, heteroaryl, and aryl groups within R₂ are each optionally substituted with at least one R₁₀₅ group;
L is —[C(R₃)(R₄)]₀₋₃—, wherein R₃ and R₄ at each occurrence are independently selected from H and alkyl;
Q is selected from formulae (IVa), (IVb), (IVc), and (IVd);
IV(a) is 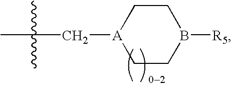
wherein A and B are independently selected from —CH— and —N—;
R₅ is selected from H, heterocycloalkyl, and heteroaryl;
(IVb) is 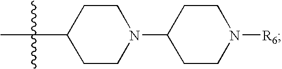
R₆ is selected from H and alkyl;
(IVc) is 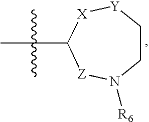
wherein the cyclic ring in formula (IIc) optionally contains at least one double bond;
wherein X is selected from —CH₂—, —N—, and —N(R₇)—;
Y is selected from —NH—, —O—, —S(O)₀₋₂—, N(R₇)(R₈), and —C(R₉)(R₁₀)—;
or X and Y together form a fused aromatic ring; and
Z is selected from —CH₂— and —C(O)—;
R₇ and R₈ each independently are selected from H and alkyl;
R₉ and R₁₀ each independently are selected from H and phenyl;
(IVd) is

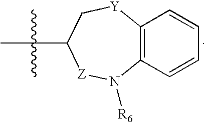

An embodiment of the present invention is a method of preventing or treating conditions which benefit from inhibition of the bradykinin B₁ receptor, comprising administering to a host in need thereof a composition comprising a therapeutically effective amount of at least one compound of formula (I).

Among the compounds of formula (I), examples include 3-Benzo[1,3]dioxol-5-yl-3-(6-methoxy-naphthalene-2-sulfonylamino)-N-[2-(3,4,5,6-tetrahydro-2H-[1,4′]bipyridinyl-4-yl)-ethyl]-propionamide, 3-Benzo[1,3]dioxol-5-yl-3-(6-methoxy-naphthalene-2-sulfonylamino)-N-(3,4,5,6-tetrahydro-2H-[1,4′]bipyridinyl-4-ylmethyl)-propionamide, 3-Benzo[1,3]dioxol-5-yl-3-(6-methoxy-naphthalene-2-sulfonylamino)-N-(2-oxo-5-phenyl-2,3-dihydro-1H-benzo[e][1,4]diazepin-3-yl)-propionamide, 3-Benzo[1,3]dioxol-5-yl-3-(6-methoxy-naphthalene-2-sulfonylamino)-N-(1-methyl-2-oxo-5-phenyl-2,3-dihydro-1H-benzo[e][1,4]diazepin-3-yl)-propionamide, 5-[(2,6-Dichloro-benzenesulfonyl)-methyl-amino]-pentanoic acid[2-(3,4,5,6-tetrahydro-2H-[1,4]bipyridinyl-4-yl)-ethyl]-amide, 5-[(2,6-Dichloro-benzenesulfonyl)-methyl-amino]-pentanoic acid (3,4,5,6-tetrahydro-2H-[1,4′]bipyridinyl-4-ylmethyl)-amide, 5-[(2,6-Dichloro-benzenesulfonyl)-methyl-amino]-pentanoic acid (2-oxo-5-phenyl-2,3-dihydro-1H-benzo[e][1,4]diazepin-3-yl)-amide, 5-[(2,6-Dichloro-benzenesulfonyl)-methyl-amino]-pentanoic acid (1-methyl-2-oxo-5-phenyl-2,3-dihydro-1H-benzo[e][1,4]diazepin-3-yl)-amide, N-[2-(3,4,5,6-Tetrahydro-2H-[1,4′]bipyridinyl-4-yl)-ethyl]-2-[1-(3-trifluoromethyl-benzenesulfonyl)-piperidin-2-yl]-acetamide, N-(3,4,5,6-Tetrahydro-2H-[1,4′]bipyridinyl-4-yl methyl)-2-[1-(3-trifluoromethyl-benzenesulfonyl)-piperidin-2-yl]-acetamide, N-(2-Oxo-5-phenyl-2,3-dihydro-1H-benzo[e][1,4]diazepin-3-yl)-2-[1-(3-trifluoromethyl-benzenesulfonyl)-piperidin-2-yl]-acetamide, N-(1-Methyl-2-oxo-5-phenyl-2,3-dihydro-1H-benzo[e][1,4]diazepin-3-yl)-2-[1-(3-trifluoromethyl-benzenesulfonyl)-piperidin-2-yl]-acetamide, 2-[1-(Naphthalene-2-sulfonyl)-3-oxo-piperazin-2-yl]-N-[2-(3,4,5,6-tetrahydro-2H-[1,4′]bipyridinyl-4-yl)-ethyl]-acetamide, 2-[1-(Naphthalene-2-sulfonyl)-3-oxo-piperazin-2-yl]-N-(3,4,5,6-tetrahydro-2H-[1,4′]bipyridinyl-4-ylmethyl)-acetamide, 2-[1-(Naphthalene-2-sulfonyl)-3-oxo-piperazin-2-yl]-N-(2-oxo-5-phenyl-2,3-dihydro-1H-benzo[e][1,4]diazepin-3-yl)-acetamide, N-(1-Methyl-2-oxo-5-phenyl-2,3-dihydro-1H-benzo[e][1,4]diazepin-3-yl)-2-[1-(naphthalene-2-sulfonyl)-3-oxo-piperazin-2-yl]-acetamide, Pyrimidine-5-carboxylic acid{1-[2-(3,4,5,6-tetrahydro-2H-[1,4′]bipyridinyl-4-yl)-ethylcarbamoyl]-cyclobutyl}-amide, Pyrimidine-5-carboxylic acid{1-[(3,4,5,6-tetrahydro-2H-[1,4′]bipyridinyl-4-ylmethyl)-carbamoyl]-cyclobutyl}-amide, Pyrimidine-5-carboxylic acid[1-(2-oxo-5-phenyl-2,3-dihydro-1H-benzo[e][1,4]diazepin-3-ylcarbamoyl)-cyclobutyl]-amide, Pyrimidine-5-carboxylic acid[1-(1-methyl-2-oxo-5-phenyl-2,3-dihydro-1H-benzo[e][1,4]diazepin-3-ylcarbamoyl)-cyclobutyl]-amide, 1-(2-Oxo-5-phenethyl-1-propyl-2,3-dihydro-1H-benzo[e][1,4]diazepin-3-yl)-3-[2-(3,4,5,6-tetrahydro-2H-[1,4′]bipyridinyl-4-yl)-ethyl]-urea, 1-(2-Oxo-5-phenethyl-1-propyl-2,3-dihydro-1H-benzo[e][1,4]diazepin-3-yl)-3-(3,4,5,6-tetrahydro-2H-[1,4′]bipyridinyl-4-ylmethyl)-urea, and the like, or pharmaceutically acceptable salts thereof.

In those cases where the compounds of formula (I) exist as tautomers, optical isomers or geometric isomers, the above formulas are intended to represent isomer mixtures as well as the individual isomeric bradykinin B₁ receptor antagonist or intermediate isomers, all of which are encompassed within the scope of this invention.

Further, references to the compounds of formula (I) with respect to pharmaceutical applications thereof are also intended to include pharmaceutically acceptable salts of the compounds of formula (I).

Exemplary formula (I) compounds are provided in Examples 1-1 through 1-22 below.

| Example | |
|---|---|
| No. | Compound |
| 1-1. | 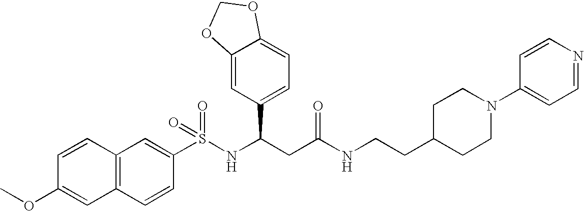 |
| | 3-Benzo[1,3]dioxol-5-yl-3-(6-methoxy- |
| | naphthalene-2-sulfonylamino)-N-[2- |
| | (3,4,5,6-tetrahydro-2H-[1,4′]bipyridinyl-4- |
| | yl)-ethyl]-propionamide |
| 1-2. | 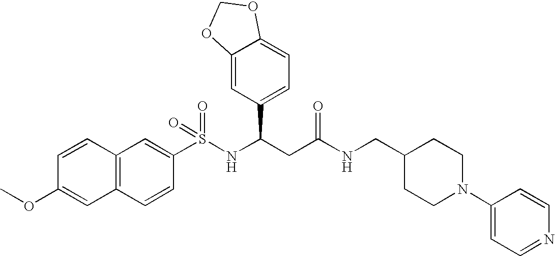 |
| | 3-Benzo[1,3]dioxol-5-yl-3-(6-methoxy- |
| | naphthalene-2-sulfonylamino)-N-(3,4,5,6- |
| | tetrahydro-2H-[1,4′]bipyridinyl-4-ylmethyl)- |
| | propionamide |
| 1-3. | 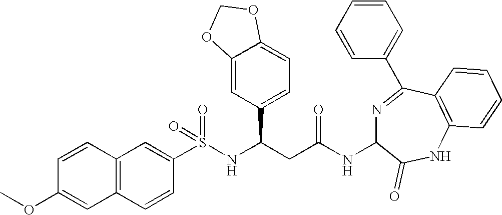 |
| | 3-Benzo[1,3]dioxol-5-yl-3-(6-methoxy- |
| | naphthalene-2-sulfonylamino)-N-(2-oxo-5- |
| | phenyl-2,3-dihydro-1H- |
| | benzo[e][1,4]diazepin-3-yl)-propionamide |
| 1-4. | 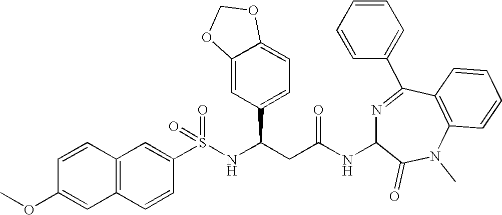 |
| | 3-Benzo[1,3]dioxol-5-yl-3-(6-methoxy- |
| | naphthalene-2-sulfonylamino)-N-(1-methyl- |
| | 2-oxo-5-phenyl-2,3-dihydro-1H- |
| | benzo[e][1,4]diazepin-3-yl)-propionamide |
| 1-5. | 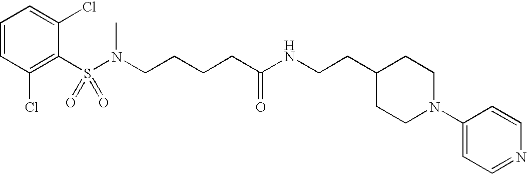 |
| | 5-[(2,6-Dichloro-benzenesulfonyl)-methyl- |
| | amino]-pentanoic acid [2-(3,4,5,6- |
| | tetrahydro-2H-[1,4′]bipyridinyl-4-yl)-ethyl]- |
| | amide |
| 1-6. | 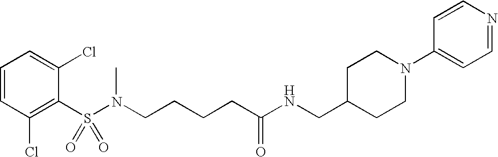 |
| | 5-[(2,6-Dichloro-benzenesulfonyl)-methyl- |
| | amino]-pentanoic acid (3,4,5,6-tetrahydro- |
| | 2H-[1,4′]bipyridinyl-4-ylmethyl)-amide |
| 1-7. | 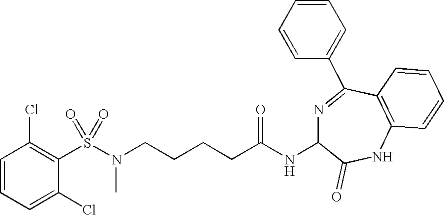 |
| | 5-[(2,6-Dichloro-benzenesulfonyl)-methyl- |
| | amino]-pentanoic acid (2-oxo-5-phenyl-2,3- |
| | dihydro-1H-benzo[e][1,4]diazepin-3-yl)- |
| | amide |
| 1-8. | 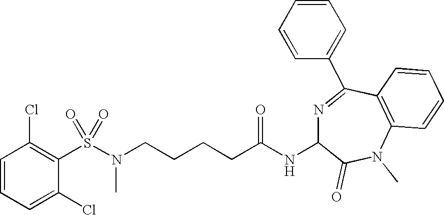 |
| | 5-[(2,6-Dichloro-benzenesulfonyl)-methyl- |
| | amino]-pentanoic acid (1-methyl-2-oxo-5- |
| | phenyl-2,3-dihydro-1H- |
| | benzo[e][1,4]diazepin-3-yl)-amide |
| 1-9. | 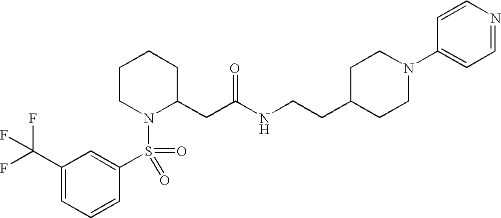 |
| | N-[2-(3,4,5,6-Tetrahydro-2H- |
| | [1,4′]bipyridinyl-4-yl)-ethyl]-2-[1-(3- |
| | trifluoromethyl-benzenesulfonyl)-piperidin |
| | 2-yl]-acetamide |
| 1-10. | 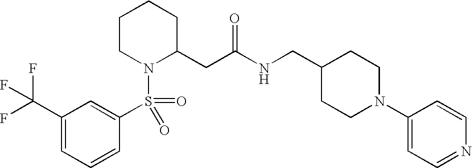 |
| | N-(3,4,5,6-Tetrahydro-2H-[1,4′]bipyridinyl- |
| | 4-ylmethyl)-2-[1-(3-trifluoromethyl- |
| | benzenesulfonyl)-piperidin-2-yl]-acetamide |
| 1-11. | 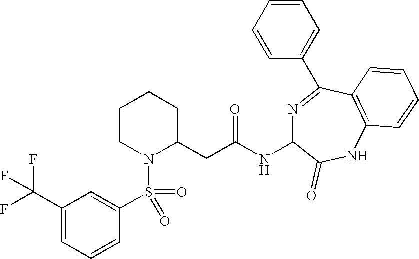 |
| | N-(2-Oxo-5-phenyl-2,3-dihydro-1H- |
| | benzo[e][1,4]diazepin-3-yl)-2-[1-(3- |
| | trifluoromethyl-benzenesulfonyl)-piperidin |
| | 2-yl]-acetamide |
| 1-12. | 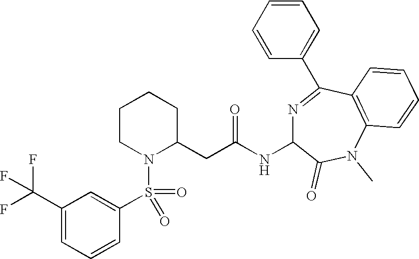 |
| | N-(1-Methyl-2-oxo-5-phenyl-2,3-dihydro- |
| | 1H-benzo[e][1,4]diazepin-3-yl)-2-[1-(3- |
| | trifluoromethyl-benzenesulfonyl)-piperidin |
| | 2-yl]-acetamide |
| 1-13. | 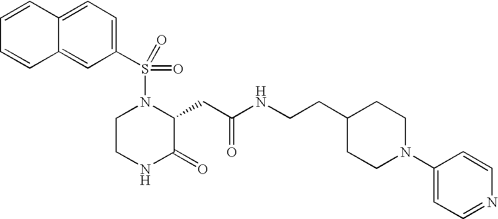 |
| | 2-[1-(Naphthalene-2-sulfonyl)-3-oxo- |
| | piperazin-2-yl]-N-[2-(3,4,5,6-tetrahydro-2H- |
| | [1,4′]bipyridinyl-4-yl)-ethyl]-acetamide |
| 1-14. | 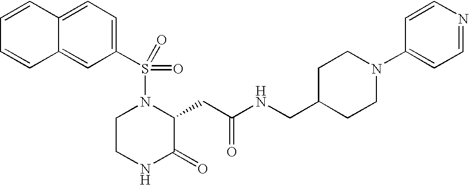 |
| | 2-[1-(Naphthalene-2-sulfonyl)-3-oxo- |
| | piperazin-2-yl]-N-(3,4,5,6-tetrahydro-2H- |
| | [1,4′]bipyridinyl-4-ylmethyl)-acetamide |
| 1-15. | 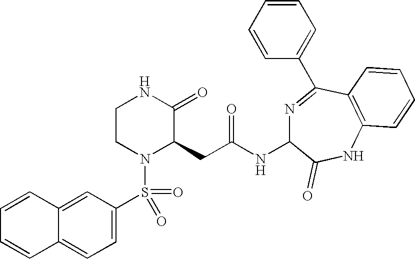 |
| | 2-[1-(Naphthalene-2-sulfonyl)-3-oxo- |
| | piperazin-2-yl]-N-(2-oxo-5-phenyl-2,3- |
| | dihydro-1H-benzo[e][1,4]diazepin-3-yl)- |
| | acetamide |
| 1-16. | 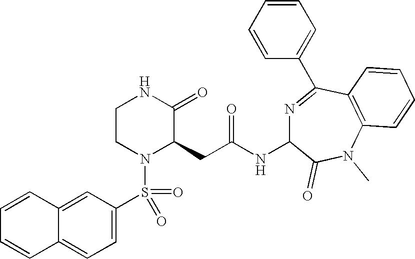 |
| | N-(1-Methyl-2-oxo-5-phenyl-2,3-dihydro- |
| | 1H-benzo[e][1,4]diazepin-3-yl)-2-[1- |
| | (naphthalene-2-sulfonyl)-3-oxo-piperazin-2- |
| | yl]-acetamide |
| 1-17. | 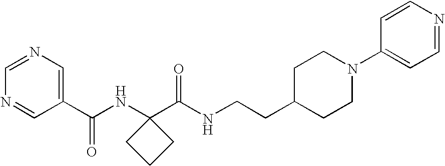 |
| | Pyrimidine-5-carboxylic acid{1-[2-(3,4,5,6- |
| | tetrahydro-2H-[1,4′]bipyridinyl-4-yl)- |
| | ethylcarbamoyl]-cyclobutyl}-amide |
| 1-18. | 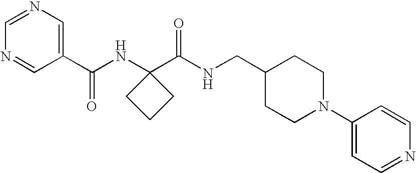 |
| | Pyrimidine-5-carboxylic acid {1-[(3,4,5,6- |
| | tetrahydro-2 H-[1,4′]bipyridinyl-4-ylmethyl)- |
| | carbamoyl]-cyclobutyl}-amide |
| 1-19. | 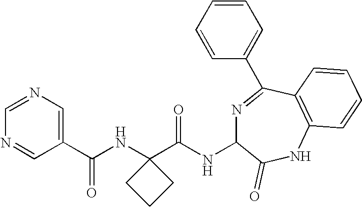 |
| | Pyrimidine-5-carboxylic acid [1-(2-oxo-5- |
| | phenyl-2,3-dihydro-1H- |
| | benzo[e][1,4]diazepin-3-ylcarbamoyl)- |
| | cyclobutyl]-amide |
| 1-20. | 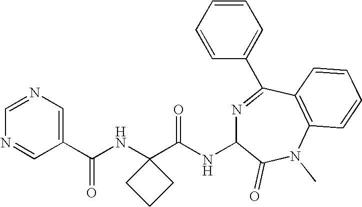 |
| | Pyrimidine-5-carboxylic acid [1-(1-methyl- |
| | 2-oxo-5-phenyl-2,3-dihydro-1H- |
| | benzo[e][1,4]diazepin-3-ylcarbamoyl)- |
| | cyclobutyl]-amide |
| 1-21. | 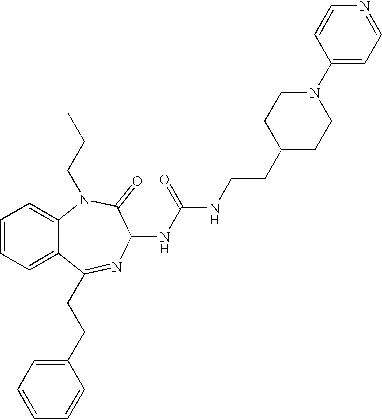 |
| | 1-(2-Oxo-5-phenethyl-1-propyl-2,3-dihydro |
| | 1H-benzo[e][1,4]diazepin-3-yl)-3-[2- |
| | (3,4,5,6-tetrahydro-2H-[1,4′]bipyridinyl-4- |
| | yl)-ethyl]-urea |
| 1-22. | 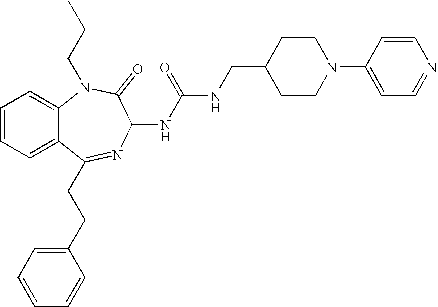 |
| | 1-(2-Oxo-5-phenethyl-1-propyl-2,3-dihydro- |
| | 1H-benzo[e][1,4]diazepin-3-yl)-3-(3,4,5,6- |
| | tetrahydro-2H-[1,4′]bipyridinyl-4-ylmethyl)- |
| | urea |

In an embodiment, the present invention provides compounds of formula (I) that are selective antagonists of bradykinin B₁ receptor over bradykinin B₂ receptor.

In another embodiment, the present invention provides a method for selectively inhibiting bradykinin B₁ receptor over bradykinin B₂ receptor comprising, using a compound of formula (I).

In another embodiment, the present invention provides a pharmaceutical composition comprising a pharmaceutically acceptable carrier and a therapeutically effective amount of a compound of formula (I) or mixtures thereof effective to treat or ameliorate adverse symptoms in mammals mediated by bradykinin B₁ receptor.

In another embodiment, the present invention provides a method for treating or ameliorating adverse symptoms in mammals mediated at least in part by bradykinin B₁ receptor comprising, administering a therapeutically effective amount of a compound of formula (I) or mixtures thereof or as is more generally the case a pharmaceutical composition.

In another embodiment, the present invention provides a pharmaceutical composition comprising a pharmaceutically acceptable carrier and a therapeutically effective amount of a compound of formula (I) or mixtures thereof to treat or ameliorate adverse symptoms in mammals associated with up-regulating bradykinin B₁ receptor following tissue damage or inflammation.

In another embodiment, the present invention provides a method for treating or ameliorating adverse symptoms in mammals associated with up-regulating bradykinin B₁ receptor following tissue damage or inflammation comprising, administering a therapeutically effective amount of a compound of formula (I), or mixtures thereof or as is more generally the case a pharmaceutical composition.

In another embodiment, the present invention provides a method for treating or ameliorating adverse symptoms associated with the presence or secretion of bradykinin B₁ receptor agonists in mammals comprising, administering a therapeutically effective amount of a compound of formula (I), or mixtures thereof or as is more generally the case a pharmaceutical composition.

In another embodiment, the present invention provides a method for treating or ameliorating pain, inflammation, septic shock or the scarring process in mammals mediated at least in part by bradykinin B₁ receptor in such mammals comprising, administering a therapeutically effective amount of a compound of formula (I), or mixtures thereof or as is more generally the case the pharmaceutical composition.

In another embodiment, the present invention provides a method for treating or ameliorating adverse symptoms associated with up-regulating bradykinin B₁ receptor relative to burns, perioperative pain, migraine, shock, central nervous system injury, asthma, rhinitis, premature labor, inflammatory arthritis, inflammatory bowel disease or neuropathic pain, comprising, administering a therapeutically effective amount of a compound of formula (I) or mixtures thereof or as is more generally the case the pharmaceutical composition.

In another embodiment, the present invention provides a method for treating or ameliorating adverse symptoms associated with the presence or secretion of bradykinin B₁ receptor agonists in mammals comprising, administering a therapeutically effective amount of a compound of formula (I) or mixtures thereof or as is more generally the case the pharmaceutical composition.

In another embodiment, the present invention provides a method for determining bradykinin B₁ receptor agonist levels in a biological sample comprising, contacting said biological sample with a compound of formula (I), at a predetermined concentration.

### Definitions

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, it is to be understood that the substituents can be the same or different.

Unless otherwise expressly defined with respect to a specific occurrence of the term, the following terms as used herein shall have the following meanings regardless of whether capitalized or not:

The term “alkyl” or the prefix “alk” in the present invention refers to straight or branched chain alkyl groups having 1 to 20 carbon atoms. An alkyl group may optionally comprise at least one double bond and/or at least one triple bond. The alkyl groups herein are unsubstituted or substituted in one or more positions with various groups. For example, such alkyl groups may be optionally substituted with at least one group selected from alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, halo alkyl, halo alkoxy, aminoalkyl, monoalkylaminoalkyl, dialkylaminoalkyl, and the like. Additionally, at least one carbon within any such alkyl may be optionally replaced with —C(O)—.

Examples of alkyls include methyl, ethyl, ethenyl, ethynyl, propyl, 1-ethyl-propyl, propenyl, propynyl, isopropyl, n-butyl, isobutyl, sec-butyl, tert-butyl, 2-methylbutyl, 3-methyl-butyl, 1-but-3-enyl, butynyl, pentyl, 2-pentyl, isopentyl, neopentyl, 3-methylpentyl, 1-pent-3-enyl, 1-pent-4-enyl, pentyn-2-yl, hexyl, 2-hexyl, 3-hexyl, 1-hex-5-enyl, formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1-dimethyl-ethyl, 1,1-dimethyl-propyl, cyano-dimethyl-methyl, propylamino, and the like.

In an embodiment, alkyls may be selected from the group comprising sec-butyl, isobutyl, ethynyl, 1-ethyl-propyl, pentyl, 3-methyl-butyl, pent-4-enyl, isopropyl, tert-butyl, 2-methylbutane, and the like.

In another embodiment, alkyls may be selected from formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1-dimethyl-ethyl, 1,1-dimethyl-propyl, cyano-dimethyl-methyl, propylamino, and the like.

In an embodiment, “alkyl” or “alk” may be selected from alkyl groups having from 1 to 6 carbon atoms with respect to the definition of “alkyl” or “alk”.

In an embodiment, an alkyl may optionally be substituted with at least one group independently selected from alkoxy, acyl, acylamino, thiocarbonylamino, acyloxy, amino, alkyl amino, amidino, alkylamidino, thioamidino, aminoacyl, aminocarbonylamino, aminothiocarbonylamino, aminocarbonyloxy, aryl, aryloxy, substituted aryloxy, aryloxylaryl, cyano, halogen, hydroxyl, nitro, oxo, thioxo, carboxyl, carboxylalkyl, carboxyl-cycloalkyl, carboxylaryl, carboxylheteroaryl, carboxylheterocyclic, cycloalkyl, guanidino, guanidinosulfone, thiol, thioalkyl, thioalkoxy, thioaryl, thiocycloalkyl, thioheteroaryl, thioheterocyclic, heteroaryl, heterocyclic, cycloalkoxy, heteroaryloxy, heterocyclyloxy, oxycarbonylamino, oxythiocarbonylamino, —OS(O)₂-alkyl, —OS(O)₂-aryl, —OS(O)₂—OS(O)₂-heteroaryl, —OS(O)₂-heterocyclic, —OSO₂—NRR where R is hydrogen or alkyl, —NRS(O)₂-alkyl, —NRS(O)₂-aryl, —NRS(O)₂-heteroaryl, —NRS(O)₂-heterocyclic, —NRS(O)₂—NR-alkyl, —NRS(O)₂—NR-aryl, —NRS(O)₂—NR-heteroaryl, and —NRS(O)₂—NR-heterocyclic, where R is hydrogen or alkyl.

The term “alkoxy” in the present invention refers to straight or branched chain alkyl groups, wherein an alkyl group is as defined above, and having 1 to 20 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexyloxy, heptyloxy, allyloxy, 2-(2-methoxy-ethoxy)-ethoxy, benzyloxy, 3-methylpentoxy, and the like.

In an embodiment, alkoxy groups may be selected from the group comprising allyloxy, hexyloxy, heptyloxy, 2-(2-methoxy-ethoxy)-ethoxy, and benzyloxy.

The term “—C(O)-alkyl” or “alkanoyl” refers to an acyl radical derived from an alkylcarboxylic acid, a cycloalkylcarboxylic acid, a heterocycloalkylcarboxylic acid, an arylcarboxylic acid, an arylalkylcarboxylic acid, a heteroarylcarboxylic acid, or a heteroarylalkylcarboxylic acid, examples of which include formyl, acetyl, 2,2,2-trifluoroacetyl, propionyl, butyryl, valeryl, 4-methylvaleryl, and the like.

The term “cycloalkyl” refers to an optionally substituted carbocyclic ring system of one or more 3, 4, 5, 6, or 7 membered rings. A cycloalkyl can further include 9, 10, 11, 12, 13, and 14 membered fused ring systems. A cycloalkyl can be saturated or partially unsaturated. A cycloalkyl may be monocyclic, bicyclic, tricyclic, and the like. Bicyclic and tricyclic as used herein are intended to include both fused ring systems, such as adamantyl, octahydroindenyl, decahydro-naphthyl, and the like, substituted ring systems, such as cyclopentylcyclohexyl and the like, and spirocycloalkyls such as spiro[2.5]octane, spiro[4.5]decane, 1,4-dioxa-spiro[4.5]decane, and the like. A cycloalkyl may optionally be a benzo fused ring system, which is optionally substituted as defined herein with respect to the definition of aryl. At least one —CH₂— group within any such cycloalkyl ring system may be optionally replaced with —C(O)—, —C(S)—, —C(═N—H)—, —C(═N—OH)—, —C(═N-alkyl)- (optionally substituted as defined herein with respect to the definition of alkyl), or —C(═N—O-alkyl)- (optionally substituted as defined herein with respect to the definition of alkyl).

Further examples of cycloalkyl radicals include cyclopropyl, cyclobutyl, cyclopentyl, cyclohexyl, octahydronaphthyl, 2,3-dihydro-1H-indenyl, and the like.

In an embodiment a cycloalkyl may be selected from the group comprising cyclopentyl, cyclohexyl, cycloheptyl, adamantenyl, bicyclo[2.2.1]heptyl, and the like.

The cycloalkyl groups herein are unsubstituted or substituted in at least one position with various groups. For example, such cycloalkyl groups may be optionally substituted with alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkylthio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like.

The term “cycloalkylcarbonyl” refers to an acyl radical of the formula cycloalkyl-C(O)— in which the term “cycloalkyl” has the significance given above, such as cyclopropylcarbonyl, cyclohexylcarbonyl, adamantylcarbonyl, 1,2,3,4-tetrahydro-2-naphthoyl, 2-acetamido-1,2,3,4-tetrahydro-2-naphthoyl, 1-hydroxy-1,2,3,4-tetrahydro-6-naphthoyl, and the like.

The term “heterocycloalkyl,” “heterocycle,” or “heterocyclyl,” refers to a monocyclic, bicyclic, or tricyclic heterocycle radical, containing at least one nitrogen, oxygen, or sulfur atom ring member and having 3, 4, 5, 6, 7, or 8 ring members in each ring, wherein at least one ring in the heterocycloalkyl ring system may optionally contain at least one double bond. At least one —CH₂— group within any such heterocycloalkyl ring system may be optionally replaced with —C(O)—, —C(S)—, —C(═N—H)—, —C(═N—OH)—, —C(═N-alkyl)-, (optionally substituted as defined herein with respect to the definition of alkyl) or —C(═N—O-alkyl)- (optionally substituted as defined herein with respect to the definition of alkyl).

The term “bicyclic” and “tricyclic” as used herein are intended to include both fused ring systems, such as 2,3-dihydro-1H-indole, and the like, and substituted ring systems, such as bicyclohexyl, and the like. At least one —CH₂— group within any such heterocycloalkyl ring system may be optionally replaced with —C(O)—, —C(N)— or —C(S)—. Heterocycloalkyl is intended to include sulfones, sulfoxides, N-oxides of tertiary nitrogen ring members, carbocyclic fused and benzo fused ring systems wherein the benzo fused ring system is optionally substituted as defined herein with respect to the definition of aryl, and the like. Such heterocycloalkyl radicals may be optionally substituted on one or more carbon atoms by halogen, alkyl, alkoxy, cyano, nitro, amino, alkylamino, dialkylamino, monoalkylaminoalkyl, dialkylaminoalkyl, haloalkyl, haloalkoxy, aminohydroxy, oxo, aryl, aralkyl, heteroaryl, heteroaralkyl, amidino, N-alkylamidino, alkoxycarbonylamino, alkylsulfonylamino, and the like, and/or on a secondary nitrogen atom (i.e., —NH—) by hydroxy, alkyl, aralkoxycarbonyl, alkanoyl, heteroaralkyl, phenyl, phenylalkyl, and the like.

Examples of a heterocycloalkyl include morpholinyl, thiomorpholinyl, thiomorpholinyl S-oxide, thiomorpholinyl S,S-dioxide, piperazinyl, homopiperazinyl, pyrrolidinyl, pyrrolinyl, 2,5-dihydro-pyrrolyl, tetrahydropyranyl, pyranyl, thiopyranyl, piperidinyl, tetrahydrofuranyl, tetrahydrothienyl, imidazolidinyl, homopiperidinyl, 1,2-dihyrdo-pyridinyl, homomorpholinyl, homothiomorpholinyl, homothiomorpholinyl S,S-dioxide, oxazolid inonyl, dihydropyrazolyl, dihydropyrrolyl, 1,4-dioxa-spiro[4.5]decyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide, 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonylpiperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

In an embodiment, a heterocycloalkyl may be selected from pyrrolidinyl, 2,5-dihydro-pyrrolyl, piperidinyl, 1,2-dihyrdo-pyridinyl, pyranyl, piperazinyl, imidazolidinyl, thiopyranyl, tetrahydropyranyl, 1,4-dioxa-spiro[4.5]decyl, and the like.

In another embodiment, a heterocycloalkyl may be selected from 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonyl piperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

The term “aryl” refers to an aromatic carbocyclic group having a single ring (e.g., phenyl) or multiple condensed rings in which at least one ring is aromatic. The aryl may be monocyclic, bicyclic, tricyclic, etc. Bicyclic and tricyclic as used herein are intended to include both fused ring systems, such as naphthyl and β-carbolinyl, and substituted ring systems, such as biphenyl, phenylpyridyl, diphenylpiperazinyl, tetrahydronaphthyl, and the like. Preferred aryl groups of the present invention include phenyl, 1-naphthyl, 2-naphthyl, indanyl, indenyl, dihydronaphthyl, fluorenyl, tetralinyl, 6,7,8,9-tetrahydro-5H-benzo[a]cycloheptenyl, and the like. The aryl groups herein are unsubstituted or substituted in one or more positions with various groups. For example, such aryl groups may be optionally substituted with alkyl, alkoxy, —C(O)H, carboxy, alkoxycarbonyl, aryl, heteroaryl, cycloalkyl, heterocycloalkyl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, aralkoxycarbonylamino, halo alkyl, halo alkoxy, aminoalkyl, monoalkylaminoalkyl, dialkylaminoalkyl, and the like. The term “aryl” further include alkaryl groups, including benzyl, 2-phenylethyl, 3-phenyl-n-propyl, and the like.

Examples of aryl groups include phenyl, naphth-2-yl, naphth-1-yl; and the like. Some preferred substituted aryl groups include monosubstituted phenyls, disubstituted phenyls and trisubstituted phenyls such as 5-dimethylaminonaphth-1-yl, 2-chlorophenyl, 2-fluorophenyl, 2-bromophenyl, 2-hydroxyphenyl, 2-nitrophenyl, 2-methylphenyl, 2-methoxyphenyl, 2-phenoxyphenyl, 2-trifluoromethylphenyl, 4-fluorophenyl, 4-chlorophenyl, 4-bromophenyl, 4-nitrophenyl, 4-methylphenyl, 4-hydroxyphenyl, 4-methoxyphenyl, 4-ethoxyphenyl, 4-butoxyphenyl, 4-iso-propylphenyl, 4-phenoxyphenyl, 4-trifluoromethylphenyl, 4-hydroxymethylphenyl, 3-methoxyphenyl, 3-hydroxyphenyl, 3-nitrophenyl, 3-fluorophenyl, 3-chlorophenyl, 3-bromophenyl, 3-phenoxyphenyl, 3-thiomethoxyphenyl, 3-methylphenyl, 3-trifluoromethylphenyl, 2,3-dichlorophenyl, 2,3-difluorophenyl, 2,4-dichlorophenyl, 2,5-dimethoxyphenyl, 3,4-dichlorophenyl, 3,4-difluorophenyl, 3,4-methylenedioxyphenyl, 3,4-dimethoxyphenyl, 3,5-difluorophenyl, 3,5-dichlorophenyl, 3,5-di-(trifluoromethyl)phenyl, 3,5-dimethoxyphenyl, 2,4-dichlorophenyl, 2,4-difluorophenyl, 2,6-difluorophenyl, 3,4,5-trifluorophenyl, 3,4,5-trimethoxyphenyl, 3,4,5-tri-(trifluoromethyl)phenyl, 2,4,6-trifluorophenyl, 2,4,6-trimethylphenyl, 2,4,6-tri-(trifluoromethyl)phenyl, 2,3,5-trifluorophenyl, 2,4,5-trifluorophenyl, 2,5-difluorophenyl, 2-fluoro-3-trifluoromethylphenyl, 4-fluoro-2-trifluoromethylphenyl, 2-fluoro-4-trifluoromethylphenyl, 4-benzyloxyphenyl, 2-chloro-6-fluorophenyl, 2,3,4,5,6-pentafluorophenyl, 2,5-dimethylphenyl, 4-phenylphenyl and 2-fluoro-3-trifluoromethylphenyl, 2-(quinolin-8-yl)sulfanylmethyl)phenyl, 2-((3-methylphen-1-ylsufanyl)methyl)phenyl, and the like.

In an embodiment, an aryl may optionally be substituted with at least one group independently selected from hydroxy, acyl, acylamino, thiocarbonylamino, acyloxy, alkyl, alkoxy, alkenyl, alkynyl, amidino, alkylamidino, thioamidino, amino, aminoacyl, aminocarbonyloxy, aminocarbonylamino, aminothiocarbonylamino, aryl, aryloxy, cycloalkoxy, heteroaryloxy, heterocyclyloxy, carboxyl, carboxylalkyl, carboxyl-cycloalkyl, carboxylaryl, carboxylheteroaryl, carboxylheterocyclic, cyano, thiol, thioalkyl, thioaryl, thioheteroaryl, thiocycloalkyl, thioheterocyclic, cycloalkyl, substituted cycloalkyl, guanidino, guanidinosulfone, halogen, nitro, heteroaryl, heterocyclic, oxycarbonylamino, oxythiocarbonylamino, —S(O)₂-alkyl, —S(O)₂-cycloalkyl, —S(O)₂— —S(O)₂-alkenyl, —S(O)₂-aryl, —S(O)₂-heteroaryl, —S(O)₂-heterocyclic, —OS(O)₂-alkyl, —OS(O)₂-aryl, —OS(O)₂-heteroaryl-OS(O)₂-heterocyclic, —OSO₂—NRR where R is hydrogen or alkyl, —NRS(O)₂-alkyl, —NRS(O)₂-aryl, —NRS(O)₂-heteroaryl, —NRS(O)₂-heterocyclic, —NRS(O)₂—NR-alkyl, —NRS(O)₂—NR-aryl, —NRS(O)₂—NR-heteroaryl, —NRS(O)₂—NR-heterocyclic, where R is hydrogen or alkyl, and wherein each of the terms is as defined herein.

Examples of aryl radicals include phenyl, p-tolyl, 4-methoxyphenyl, 4-(tert-butoxy)phenyl, 3-methyl-4-methoxyphenyl, 4-CF₃-phenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, piperazinylphenyl, and the like.

Further examples of aryl radicals include 3-tert-butyl-1-fluoro-phenyl, 1,3-difluoro-phenyl, (1-hydroxy-1-methyl-ethyl)-phenyl, 1-fluoro-3-(2-hydroxy-1,1-dimethyl-ethyl)-phenyl, (1,1-dimethyl-propyl)-phenyl, cyclobutyl-phenyl, pyrrolidin-2-yl-phenyl, (5-oxo-pyrrolidin-2-yl)-phenyl, (2,5-dihydro-1H-pyrrol-2-yl)-phenyl, (1H-pyrrol-2-yl)-phenyl, (cyano-dimethyl-methyl)-phenyl, tert-butyl-phenyl, 1-fluoro-2-hydroxy-phenyl, 1,3-difluoro-4-propylamino-phenyl, 1,3-difluoro-4-hydroxy-phenyl, 1,3-difluoro-4-ethylamino-phenyl, 3-isopropyl-phenyl, (3H-[1,2,3]triazol-4-yl)-phenyl, [1,2,3]triazol-1-yl-phenyl, [1,2,4]thiadiazol-3-yl-phenyl, [1,2,4]thiadiazol-5-yl-phenyl, (4H-[1,2,4]triazol-3-yl)-phenyl, [1,2,4]oxadiazol-3-yl-phenyl, imidazol-1-yl-phenyl, (3H-imidazol-4-yl)-phenyl, [1,2,4]triazol-4-yl-phenyl, [1,2,4]oxadiazol-5-yl-phenyl, isoxazol-3-yl-phenyl, (1-methyl-cyclopropyl)-phenyl, isoxazol-4-yl-phenyl, isoxazol-5-yl-phenyl, 1-cyano-2-tert-butyl-phenyl, 1-trifluoromethyl-2-tert-butyl-phenyl, 1-chloro-2-tert-butyl-phenyl, 1-acetyl-2-tert-butyl-phenyl, 1-tert-butyl-2-methyl-phenyl, 1-tert-butyl-2-ethyl-phenyl, 1-cyano-3-tert-butyl-phenyl, 1-trifluoromethyl-3-tert-butyl-phenyl, 1-chloro-3-tert-butyl-phenyl, 1-acetyl-3-tert-butyl-phenyl, 1-tert-butyl-3-methyl-phenyl, 1-tert-butyl-3-ethyl-phenyl, 4-tert-butyl-1-imidazol-1-yl-phenyl, ethylphenyl, isobutylphenyl, isopropylphenyl, 3-allyloxy-1-fluoro-phenyl, (2,2-dimethyl-propyl)-phenyl, ethynylphenyl, 1-fluoro-3-heptyloxy-phenyl, 1-fluoro-3-[2-(2-methoxy-ethoxy)-ethoxy]-phenyl, 1-benzyloxy-3-fluoro-phenyl, 1-fluoro-3-hydroxy-phenyl, 1-fluoro-3-hexyloxy-phenyl, (4-methyl-thiophen-2-yl)-phenyl, (5-acetyl-thiophen-2-yl)-phenyl, furan-3-yl-phenyl, thiophen-3-yl-phenyl, (5-formyl-thiophen-2-yl)-phenyl, (3-formyl-fu ran-2-yl)-phenyl, acetylamino-phenyl, trifluoromethylphenyl, sec-butyl-phenyl, pentylphenyl, (3-methyl-butyl)-phenyl, (1-ethyl-propyl)-phenyl, cyclopentyl-phenyl, 3-pent-4-enyl-phenyl, phenyl propionic acid ethyl ester, pyridin-2-yl-phenyl, (3-methyl-pyridin-2-yl)-phenyl, thiazol-2-yl-phenyl, (3-methyl-thiophen-2-yl)-phenyl, fluoro-phenyl, adamantan-2-yl-phenyl, 1,3-difluoro-2-hydroxy-phenyl, cyclopropyl-phenyl, 1-bromo-3-tert-butyl-phenyl, (3-bromo-[1,2,4]thiadiazol-5-yl)-phenyl, (1-methyl-1H-imidazol-2-yl)-phenyl, (3,5-dimethyl-3H-pyrazol-4-yl)-phenyl, (3,6-dimethyl-pyrazin-2-yl)-phenyl, (3-cyano-pyrazin-2-yl)-phenyl, thiazol-4-yl-phenyl, (4-cyano-pyridin-2-yl)-phenyl, pyrazin-2-yl-phenyl, (6-methyl-pyridazin-3-yl)-phenyl, (2-cyano-thiophen-3-yl)-phenyl, (2-chloro-thiophen-3-yl)-phenyl, (5-acetyl-thiophen-3-yl)-phenyl, cyano-phenyl, and the like.

Alkyl, and cycloalkyl groups include, by way of example, iso-propyl, n-propyl, n-butyl, iso-butyl, sec-butyl, t-butyl, —CH₂CH═CH₂, —CH₂CH═CH(CH₂)₄CH₃, cyclopropyl, cyclobutyl, cyclohexyl, cyclopentyl, cyclohex-1-enyl, —CH₂-cyclopropyl, —CH₂-cyclobutyl, —CH₂-cyclohexyl, —CH₂-cyclopentyl, —CH₂CH₂-cyclopropyl, —CH₂CH₂-cyclobutyl, —CH₂CH₂-cyclohexyl, —CH₂CH₂-cyclopentyl, and the like.

The term “heteroaryl” refers to an aromatic heterocycloalkyl radical as defined above. The heteroaryl groups herein are unsubstituted or substituted in at least one position with various groups. For example, such heteroaryl groups may be optionally substituted with, for example, alkyl, alkoxy, halogen, hydroxy, cyano, nitro, amino, monoalkylamino, dialkylamino, haloalkyl, haloalkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, alkyl thio, alkylsulfinyl, alkylsulfonyl, aralkoxycarbonylamino, aminoalkyl, monoalkylaminoalkyl, dialkylaminoalkyl, and the like.

Examples of heteroaryl groups include pyridyl, pyrimidyl, furanyl, imidazolyl, thienyl, oxazolyl, thiazolyl, pyrazinyl, 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, quinolinyl, benzothienyl, indolyl, indolinyl, pyridazinyl, isoindolyl, isoquinolyl, quinazolinyl, quinoxalinyl, phthalazinyl, imidazolyl, isoxazolyl, pyrazolyl, indolizinyl, indazolyl, benzothiazolyl, benzimidazolyl, benzofuranyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, tetrahydrocarbazole, tetrahydrobetacarboline, and the like.

Further examples of heteroaryl include, by way of example, pyrid-2-yl, pyrid-3-yl, pyrid-4-yl, fluoropyridyls (including 5-fluoropyrid-3-yl), chloropyridyls (including 5-chloropyrid-3-yl), thiophen-2-yl, thiophen-3-yl, benzothiazol-4-yl, 2-phenylbenzoxazol-5-yl, furan-2-yl, benzofuran-2-yl, thionaphthen-2-yl, 2-chlorothiophen-5-yl, 3-methylisoxazol-5-yl, 2-(thiophenyl)thiophen-5-yl, 6-methoxythionaphthen-2-yl, 3-phenyl-1,2,4-thiooxadiazol-5-yl, 2-phenyloxazol-4-yl, 5-chloro-1,3-dimethylpyrazol-4-yl; 2-methoxycarbonyl-thiophen-3-yl; 2,3-dimethylimidazol-5-yl; 2-methylcarbonylamino-4-methyl-thiazol-5-yl; quinolin-8-yl; thiophen-2-yl; 1-methylimidiazol-4-yl; 3,5-dimethylisoxazol-4-yl; and the like.

In an embodiment, a heteroaryl group may be selected from pyridyl, pyrimidyl, furanyl, imidazolyl, thienyl, oxazolyl, thiazolyl, pyrazinyl, and the like.

In another embodiment, a heteroaryl group may be selected from 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, and the like.

Further examples of heterocycloalkyls and heteroaryls may be found in Katritzky, A. R. et al., Comprehensive Heterocyclic Chemistry: The Structure, Reactions, Synthesis and Use of Heterocyclic Compounds, Vol. 1-8, New York: Pergamon Press, 1984.

The term “aralkoxycarbonyl” refers to a radical of the formula aralkyl-O—C(O)— in which the term “aralkyl” is encompassed by the definitions above for aryl and alkyl. Examples of an aralkoxycarbonyl radical include benzyloxycarbonyl, 4-methoxyphenylmethoxycarbonyl, and the like.

The term “aryloxy” refers to a radical of the formula —O-aryl in which the term aryl is as defined above.

The term “aralkanoyl” refers to an acyl radical derived from an aryl-substituted alkanecarboxylic acid such as phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The term “aroyl” refers to an acyl radical derived from an arylcarboxylic acid, “aryl” having the meaning given above. Examples of such aroyl radicals include substituted and unsubstituted benzoyl or naphthoyl such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “haloalkyl” refers to an alkyl radical having the meaning as defined above wherein one or more hydrogens are replaced with a halogen. Examples of such haloalkyl radicals include chloromethyl, 1-bromoethyl, fluoromethyl, difluoromethyl, trifluoromethyl, 1,1,1-trifluoroethyl, and the like.

The term “epoxide” refers to chemical compounds or reagents comprising a bridging oxygen wherein the bridged atoms are also bonded to one another either directly or indirectly. Examples of epoxides include epoxyalkyl (e.g., ethylene oxide and 1,2-epoxybutane), epoxycycloalkyl (e.g., 1,2-epoxycyclohexane and 1,2-epoxy-1-methylcyclohexane), and the like.

The term “structural characteristics” refers to chemical moieties, chemical motifs, and portions of chemical compounds. These include R groups, such as those defined herein, ligands, appendages, and the like. For example, structural characteristics may be defined by their properties, such as, but not limited to, their ability to participate in intermolecular interactions including Van der Waal's interactions (e.g., electrostatic interactions, dipole-dipole interactions, dispersion forces, hydrogen bonding, and the like). Such characteristics may have an increased ability to cause the desired effect and thus prevent or treat the targeted diseases or conditions.

The term “halo” or “halogen” refers to fluoro, chloro, bromo or iodo.

The term “oxo” refers to an oxygen atom bound to an atom such as, but not limited to, carbon or nitrogen, through a double bond.

In an embodiment, examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholino, thiomorpholino, piperidinyl, pyrrolidine, tetrahydrofuranyl, and the like.

Amino acid refers to any of the naturally occurring amino acids, as well as synthetic analogs (e.g., D-stereoisomers of the naturally occurring amino acids, such as D-threonine, and L-stereoisomers of amino acids in proteins) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., glycine), alkyl (e.g., alanine, valine, leucine, isoleucine, proline), substituted alkyl (e.g., threonine, serine, methionine, cysteine, aspartic acid, asparagine, glutamic acid, glutamine, arginine, and lysine), arylalkyl (e.g., phenylalanine and tryptophan), substituted arylalkyl (e.g., tyrosine), and heteroarylalkyl (e.g., histidine). Unnatural amino acids are also known in the art, as set forth in, for example, Williams (ed.), Synthesis of Optically Active α-Amino Acids, Pergamon Press (1989); Evans et al., J. Amer. Chem. Soc., 112:4011-4030 (1990); Pu et al., J. Org Chem., 56:1280-1283 (1991); Williams et al., J. Amer. Chem. Soc., 113:9276-9286 (1991); and all references cited therein. The present invention includes the side chains of unnatural amino acids as well.

“Pharmaceutically acceptable salt” refers to pharmaceutically acceptable salts of a compound of formula (I) which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

In the examples below, the following abbreviations have the following meanings. If an abbreviation is not defined, it has its generally accepted meaning.
aq or aq.=aqueous
AcOH=acetic acid
Bd=broad doublet
bm=broad multiplet
bs=broad singlet
Bn=benzyl
Boc=N-tert-butoxylcarbonyl
Boc₂O=di-tert-butyl dicarbonate
BOP=benzotriazol-1-yloxy-tris(dimethylamino)phosphonium hexafluorophosphate
Cbz=carbobenzyloxy
CHCl₃=chloroform
CH₂Cl₂=dichloromethane
(COCl)₂=oxalyl chloride
d=doublet
dd=doublet of doublets
dt=doublet of triplets
DBU=1,8-diazabicyclo[5.4.0]undec-7-ene
DCC=1,3-dicyclohexylcarbodiimide
DMAP=4-N,N-dimethylaminopyridine
DME=ethylene glycol dimethyl ether
DMF=N,N-dimethylformamide
DMSO=dimethylsulfoxide
EDC=1-(3-dimethylaminopropyl)-3-ethylcarbodiimide hydrochloride
Et₃N=triethylamine
Et₂O=diethyl ether
EtOAc=ethyl acetate
EtOH=ethanol
eq or eq.=equivalent
Fmoc=N-(9-fluorenylmethoxycarbonyl)
FmocONSu=N-(9-fluorenylmethoxycarbonyl)-succinimide
g=gram(s)
h=hour(s)
H₂O=water
HBr=hydrobromic acid
HCl=hydrochloric acid
HOBT=1-hydroxybenzotriazole hydrate
Hr=hour
K₂CO₃=potassium carbonate
L=liter
m=multiplet
MeOH=methanol
Mg=milligram
MgSO₄=magnesium sulfate
mL=milliliter
mm=millimeter
mM=millimolar
mmol=millimol
mp=melting point
N=normal
NaCl=sodium chloride
Na₂CO₃=sodium carbonate
NaHCO₃=sodium bicarbonate
NaOEt=sodium ethoxide
NaOH=sodium hydroxide
NH₄Cl=ammonium chloride
NMM=N-methylmorpholine
Phe=L-phenylalanine
Pro=L-proline
Psi=pounds per square inch
PtO₂=platinum oxide
q=quartet
quint=quintet
rt=room temperature
s=singlet
sat=saturated
t=triplet
t-BuOH=tert-butanol
TFA=trifluoroacetic acid
THF=tetrahydrofuran
TLC=or tlc=thin layer chromatography
Ts=tosyl
TsCl=tosyl chloride
TsOH=toluene sulfonic acid
μL=microliter

### Compound Preparation

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

In the following examples and procedures, the term “Aldrich” indicates that the compound or reagent used in the procedure is commercially available from Aldrich Chemical Company, Inc., Milwaukee, Wis. 53233 USA; the term “Sigma” indicates that the compound or reagent is commercially available from Sigma, St. Louis Mo. 63178 USA; the term “TCI” indicates that the compound or reagent is commercially available from TCI America, Portland Oreg. 97203; the term “Frontier” or “Frontier Scientific” indicates that the compound or reagent is commercially available from Frontier Scientific, Utah, USA; the term “Bachem” indicates that the compound or reagent is commercially available from Bachem, Torrance, Calif., USA. The term “Matrix” or “Matrix Scientific” indicates that the compound or reagent is commercially available from Matrix Scientific, Columbia, S.C., USA. The term “Ambinter” indicates that the compound or reagent is commercially available from Ambinter Paris, France.

### General Procedure 1

### Preparation of 2-(3,4,5,6-Tetrahydro-2H-[1,4′]bipyridinyl-4-yl)-ethylamine (7)

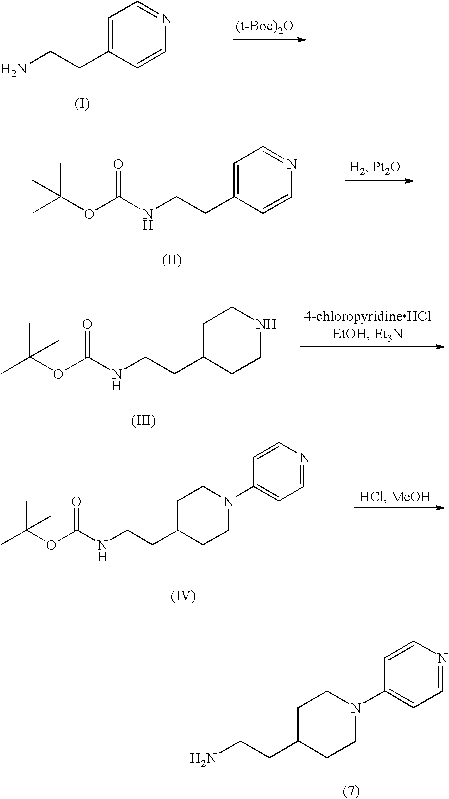

(2-Pyridin-4-yl-ethyl)-carbamic acid tert-butyl ester (II). A flask was charged with 1.1 eq. of (t-Boc)₂O and placed in an ice bath as 1.0 eq. of 2-pyridin-4-yl-ethylamine (I) (Matrix Scientific, 8218) was added drop wise (Caution: exotherm and vigorous gas evolution). After addition, the bath was removed and the reaction mixture was allowed to stir at rt for a time sufficient for reaction completion. The product was then vacuum distilled from the reaction mixture to afford (II).

(2-Piperidin-4-yl-ethyl)-carbamic acid tert-butyl ester (III). A solution of 1.0 eq. of pyridine (II) in acetic acid was prepared and 0.1 eq. of PtO₂ was added. The mixture was hydrogenated at about 10-60 psi of hydrogen for a time sufficient for reaction completion. Filtration of the mixture through Celite and removal of the solvent afforded piperidine (III) of sufficient purity for further elaboration.

[2-(3,4,5,6-Tetrahydro-2H-[1,4′]bipyridinyl-4-yl)-ethyl]-carbamic acid tert-butyl ester (IV). A solution of 1.0 eq. of piperidine (III), 1.0 eq. of 4-chloropyridine hydrochloride (Aldrich, C70223) and 2.2 eq. of triethylamine in ethanol was refluxed for a time sufficient for reaction completion. The product was isolated by column chromatography on silica gel using EtOAc as eluant to give (IV).

Preparation of the title compound (7). Piperidine (IV) was dissolved in a mixture of EtOAc and EtOH and HCl was bubbled through the solution. The reaction mixture was stirred for a time sufficient for reaction completion and concentrated. The crude material was triturated with EtOAc to afford 7 as its hydrochloride salt.

### General Procedure 2

### Preparation of 2-(3,4,5,6-Tetrahydro-2H-[1,4′]bipyridinyl-4-yl)-ethylamine (13)

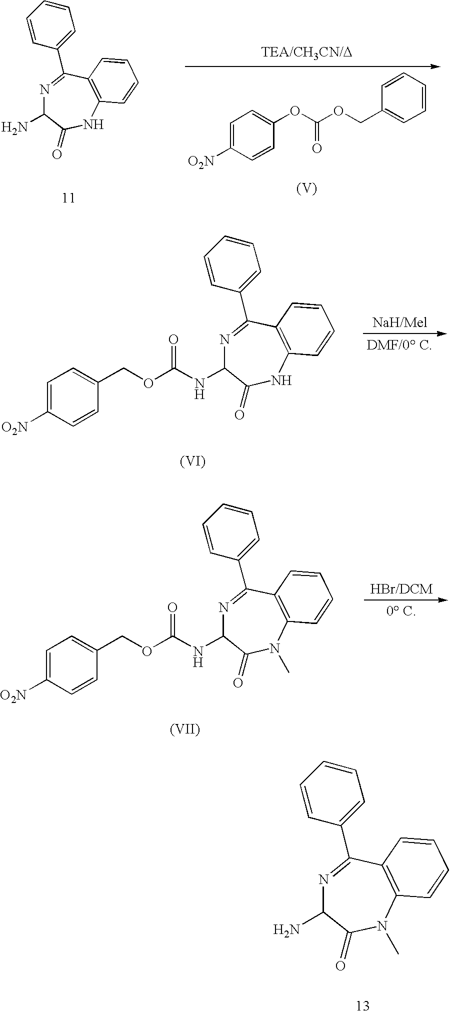

(2-Oxo-5-phenyl-2,3-dihydro-1H-benzo[e][1,4]diazepin-3-yl)-carbamic acid benzyl ester (VI). In accordance with J. Org. Chem., 60:730-734 (1995). Compound 11 (10 mmol, Tyger Scientific, A25300), carbonic acid benzyl ester 4-nitro-phenyl ester (V) (10 mmol, Aldrich, 277673), and triethylamine (10 mmol) are combined in acetonitrile (20 mL) under nitrogen and refluxed 15 hours. The reaction is concentrated, the residue dissolved in ethyl acetate (50 mL) and washed twice with 1N NaOH (50 mL). The combined aqueous layers are extracted with ethyl acetate and the combined organic layers are washed with brine, dried with sodium sulfate, and concentrated. The residue is purified via silica gel chromatography using gradients of hexanes and ethyl acetate.

(1-Methyl-2-oxo-5-phenyl-2,3-dihydro-1H-benzo[e][1,4]diazepin-3-yl)-carbamic acid benzyl ester (VII). In accordance with J. Org. Chem., 60:730-734 (1995). Compound (VI) (1 mmol) is dissolved with anhydrous DMF and cooled to 0° C. in an ice bath under nitrogen. Sodium hydride (60% dispersion in mineral oil) is added to reaction and stirred for 2 hours. Methyl iodide (1 mmol) is added and the reaction is stirred for another 2 hours under nitrogen at 0° C. The reaction is quenched by slowly pipetting it into a sat. sodium hydrogen sulfate solution (50 mL). The mixture is filtered and the resulting solid is washed with water and dried in vacuo. Once dried, the solid is dissolved in dichloromethane, dried with sodium sulfate, and concentrated. The residue is purified via silica gel chromatography using gradients of hexanes and ethyl acetate.

Preparation of the title compound (13). In accordance with J. Org. Chem., 60:730-734 (1995). A solution of compound (VII) (1 mmol) in dichloromethane (15 mL) is saturated with hydrogen bromide. After 30 minutes the reaction is concentrated in vacuo and dissolved in dichloromethane (10 mL). The product is precipitated by addition of diethyl ether and the resulting slurry is filtered. The collected solid is dried in vacuum oven.

### EXAMPLE 1

### 3-Benzo[1,3]dioxol-5-yl-3-(6-methoxy-naphthalene-2-sulfonylamino)-N-[2-(3,4,5,6-tetrahydro-2H-[1,4′]bipyridinyl-4-yl)-ethyl]-propionamide (8)

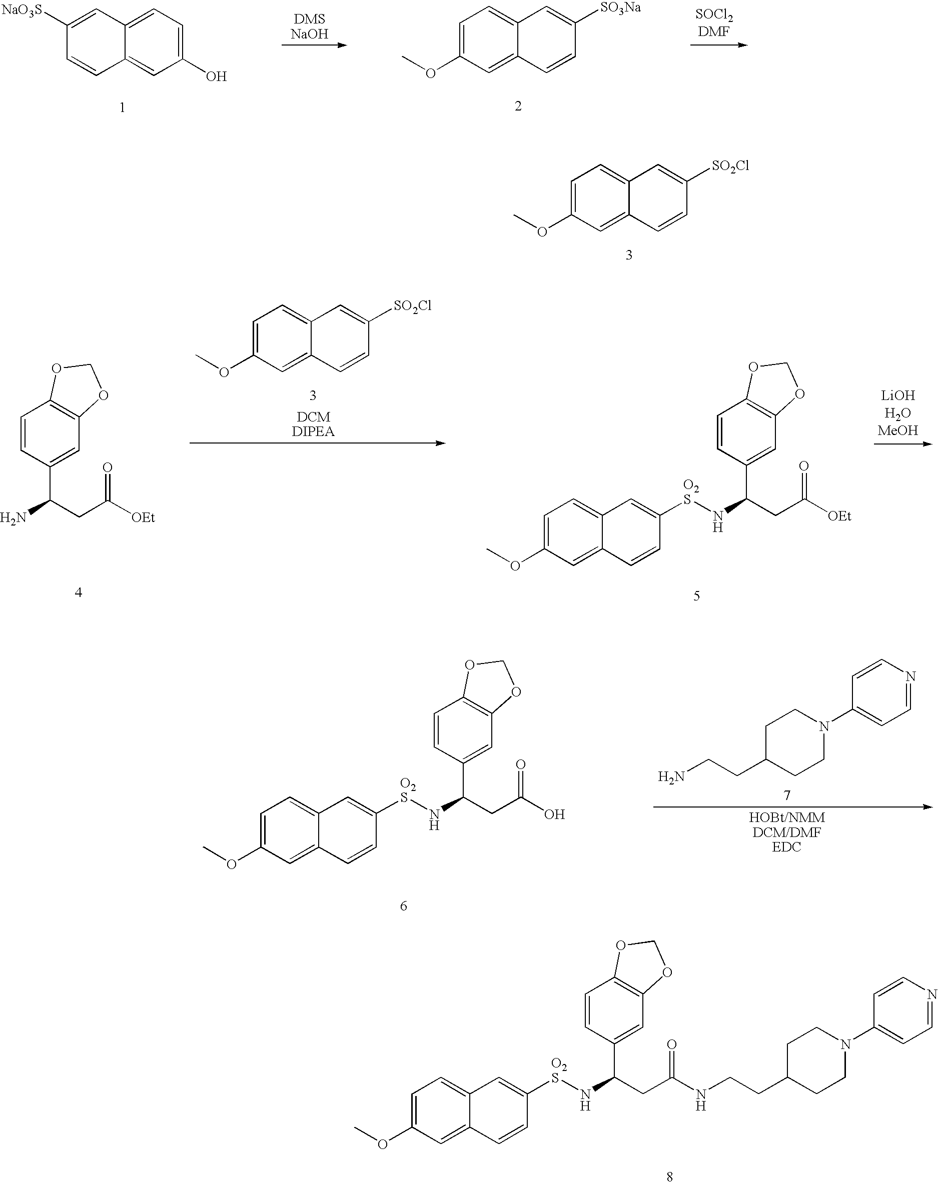

Sodium 6-methoxynaphthalene-2-sulfonate (2). The sodium 6-hydroxynaphthalene-2-sulfonate (1, 60 g, 0.25 mol; tech., 90% pure) was slurried in deionized water (600 mL) and was treated with 50% aq. NaOH (43 g, 0.52 mol). The resulted solution was stirred at 40° C. and treated with dimethyl sulfate (52 mL, 0.54 mol), drop wise over 90 minutes. Temperature throughout addition was maintained at 50-60° C. After completion of addition the mixture was stirred for 10 minutes and then NaCl (105 g) was added. Stirring was continued for additional 2 hours during which temperature was gradually lowered to ambient. The precipitated solids were filtered off and washed on the filter with brine (2×200 mL). The filter cake was washed with toluene (2×100 mL) and dried under reduced pressure (75 mm_{Hg}, 60° C.) for 80 hours. The yield of product 2 was 56 g (86%; light-beige powder).

2: 1H-NMR (DMSO-d6) δ: 8.08 (s, 1H), 7.86 (d, 1H J=9 Hz), 7.73 (m, 2H), 7.30 (d, 1H, J=2.4 Hz), 7.17 (dd, 1H, J₁=9 Hz, J₂=2.4 Hz), 3.86 (s, 3H); 13C-NMR (DMSO-d6) δ: 158.04, 143.63, 134.47, 130.25, 127.68, 126.46, 124.46, 124.40, 119.14, 106.21, 55.53.

6-Methoxynaphthalene-2-sulfonyl chloride (3). Compound 2 (51 g, 0.2 mol) was divided in two approximately equal portions. First portion was slurried in anhydrous DMF (50 mL) and cooled in an ice bath to approx. +7° C. forming a thick paste. The thionyl chloride (10 mL) was cautiously added and reaction mixture became stirrable. At this moment the remaining portion of salt 2 was added in small portions (approx. 5 g each) alternating with remaining portion of thionyl chloride (ca. 3 mL each of the remaining amount; total amount added 26 mL, 41 g, 0.34 mol). At the end the small amount of DMF was added to reduce viscosity of reaction mixture and then the cooling bath was removed. The stirring was continued for additional 3 hr at ambient temperature before pouring the reaction mixture into the ice-water mixture (500 mL). The precipitated solids were filtered off and washed with ice-cold water. The filter cake was dissolved in several portions of dichloromethane (500 mL total). The aqueous phase was separated and discarded. The organic solution was dried over anhydrous sodium sulfate, filtered through pad of Florisil (1×2 inch) using additional dichloromethane (150 mL) to rinse the pad. The combined filtrates were evaporated to give yellow solid. The crude reaction product was dissolved in MTBE (100 mL) with heating and resulted solution was left standing. Precipitated crystalline product was filtered off, rinsed with a small amount (ca. 30 mL) of cold MTBE-hexane (1:1) mixture and dried to give sulfonyl chloride 3, 32 g (63%; yellow crystals).

3: 1H-NMR (CDCl₃) δ: 8.05 (d, 1H, J=1.5 Hz), 7.94 (m, 3H), 7.38 (dd, 1H, J₁=9 Hz, J₂=2.4 Hz), 7.23 (d, 1H, J=2.4 Hz), 4.00 (s, 3H); 13C-NMR (CDCl₃) δ: 161.06, 138.66, 137.72, 131.26, 128.65, 128.41, 126.81, 121.95, 121.27, 105.94, 55.48.

(3R)-Ethyl 3-(benzo[d][1,3]dioxol-5-yl)-3-(2-methoxynaphthalene-6-sulfonamido)propanoate (5). The (3R)-ethyl 3-amino-3-(benzo[d][1,3]dioxol-5-yl)propanoate hydrochloride (4, 1.494 g, 6 mmol; Biocatalytics) was dissolved in dichloromethane (60 mL) and diisopropylethyl amine (5 mL, 5 eq) was added. The resulted mixture was stirred at ambient temperature for 5 minutes then sulfonyl chloride 3 (1.626 g, 6 mmol) was added. The resulted mixture was stirred at ambient temperature for 18 hours then was divided between ethyl acetate (500 mL) and 0.2N citric acid (200 mL). The organic layer was separated and washed with 0.2N citric acid (2×100 mL), water (1×100 mL), sat. aq. bicarbonate (2×100 mL), brine (1×100 mL) and dried with magnesium sulfate. The solution was filtered and evaporated to give colorless oil, 2.651 g (5, 97%; used in next reaction without further purification).

5: 1H-NMR (CDCl₃) δ 8.07 (s, 1H), 7.70 (m, 3H), 7.23 (dd, 1H, J₁=9 Hz, J₂=2.4 Hz), 7.14 (d, 1H, J=2.1 Hz), 6.55 (m, 3H), 5.91 (d, 1H, J=7.2 Hz), 5.74 (d, 1H, J=1.2 Hz), 5.57 (d, 1H, J=1.2 Hz), 4.72 (dd, 1H, J₁=13 Hz, J₂=6.6 Hz), 4.03 (q, 2H, J=7 Hz), 3.96 (s, 3H), 2.75 (ddd, 2H, J₁=6.6 Hz, J₂=15.6 Hz, J₃=13 Hz), 1.15 (t, 3H, J=7 Hz); 13C-NMR (CDCl₃) δ 170.53, 159.64, 147.38, 146.80, 136.16, 134.60, 132.67, 130.54, 128.27, 127.59, 127.05, 122.85, 120.17, 120.06, 107.72, 106.76, 105.50, 100.84, 60.85, 55.34, 54.36, 41.19, 13.87.

(3R)-3-(Benzo[d][1,3]dioxol-5-yl)-3-(2-methoxynaphthalene-6-sulfonamido)propanoic acid (6). The ethyl ester 5 (2.3 g, 5 mmol) was dissolved in THF (25 mL) with stirring at ambient temperature and then was treated with solution of LiOH monohydrate (2.3 g, 5.5 mmol) in water (20 mL) and methanol (100 mL). The resulted solution was stirred for 20 hr, evaporated, diluted with water (50 mL) and acidified with 1N HCl to pH3 and extracted with ethyl acetate (3×100 mL). The combined extracts were washed with brine (1×100 mL) and dried with anhydrous magnesium sulfate. The solution was filtered and filtrate evaporated to dryness to give light beige solid, 2.1 g (99%) that was used directly without further purification.

Preparation of the title compound (8). The carboxylic acid 6 (0.262 g, 0.61 mmol) was dissolved in dichloromethane (5 mL) and stirred under nitrogen at 0° C. To this mixture was added 7 (0.125 g, 0.61 mmol, prepared via general procedure 1), HOBt (0.165 g, 1.22 mmol), and N-methylmorpholine (0.33 mL, 3.05 mmol). DMF (1 mL) was added to solubilize reaction contents. This mixture was stirred at 0° C. for 30 minutes to 1 hour followed by addition of EDC (0.234 g, 1.22 mmol). The reaction was stirred overnight and allowed to come to room temperature. The contents were stripped down and taken up in equal portions of water and CHCl₃/IPA (4:1). The aqueous layer was discarded and the organic layer was washed with sat. NaHCO₃, (25 mL), H₂O (25 mL), brine (25 mL), and dried with sodium sulfate. The organics were removed via rotary evaporation and 217 mgs of a glassy solid was purified by reverse-phase HPLC. Collected 106.7 mg of 8 (28%) as TFA salt.

8: 1H-NMR (CD₃OD) δ 8.04 (d, 2H, J=7.7 Hz), 7.91 (m, 2H), 7.71 (d, 1H, J=9.2 Hz), 7.56 (s, 1H, NH), 7.53 (s, 1H, NH), 7.26 (s, 1H), 7.20 (m, 2H), 7.09 (d, 2H, 7.7 Hz), 6.51 (d, 1H, J=8 Hz), 6.38 (m, 2H), 5.58 (t, 1H, J=1.5 Hz), 5.38 (t, 1H, J=1.5 Hz), 4.74 (t, 1H, J=7.7 Hz), 4.19 (br d, 2H, J=113.4 Hz), 3.93 (s, 3H), 3.16 (m, 4H), 2.54 (m, 2H), 1.86 (t, 2H, J=15 Hz), 1.59 (br s, 1H), 1.32 (m, 2H), 1.14 (m, 2H).

### EXAMPLE 2

### 3-Benzo[1,3]dioxol-5-yl-3-(6-methoxy-naphthalene-2-sulfonylamino)-N-(3,4,5,6-tetrahydro-2H-[1,4′]bipyridinyl-4-ylmethyl)-propionamide (10)

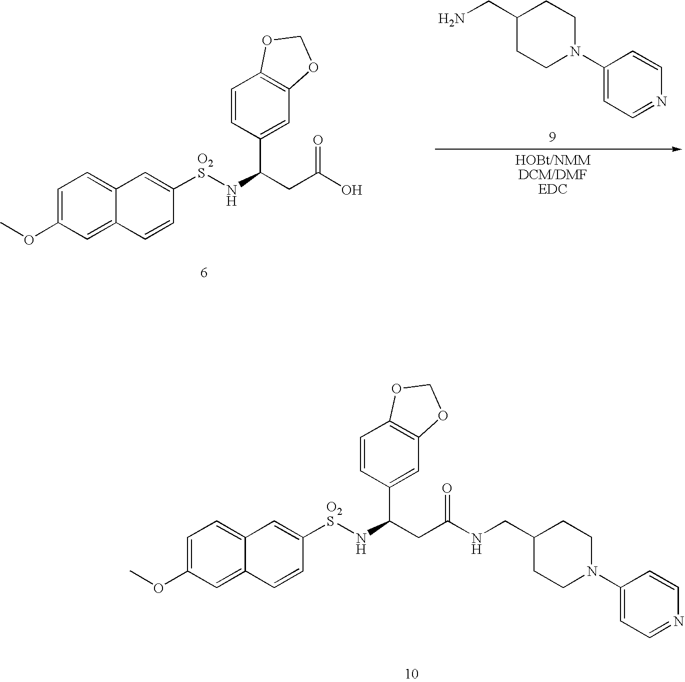

2-(3,4,5,6-Tetrahydro-2H-[1,4′]bipyridinyl-4-yl)-methylamine (9). This compound was prepared in an analogous fashion to 7 (see general procedure 1) substituting 4-picolylamine (Aldrich, A65603) for compound (I).

Preparation of the title compound (10). The carboxylic acid 6 (1 mmol) is dissolved in dichloromethane (10 mL) and stirred under nitrogen at 0° C. To this mixture is added 2-(3,4,5,6-tetrahydro-2H-[1,4′]bipyridinyl-4-yl)-methylamine 9 (1 mmol), HOBt (2 mmol), and N-methylmorpholine (5 mmol). DMF (2 mL) is added to solubilize reaction contents. This mixture is stirred at 0° C. for 30 minutes to 1 hour followed by addition of EDC (2 mmol). The reaction is stirred overnight and allowed to come to room temperature. The contents are stripped down and taken up in equal portions of water and CHCl₃/IPA (4:1). The aqueous layer is discarded and the organic layer is washed with sat. NaHCO₃, (25 mL), H₂O (25 mL), brine (25 mL), and dried with sodium sulfate. The organics are removed via rotary evaporation and 10 is purified by reverse-phase HPLC.

### EXAMPLE 3

### 3-Benzo[1,3]dioxol-5-yl-3-(6-methoxy-naphthalene-2-sulfonylamino)-N-(2-oxo-5-phenyl-2,3-dihydro-1H-benzo[e][1,4]diazepin-3-yl)-propionamide (12)

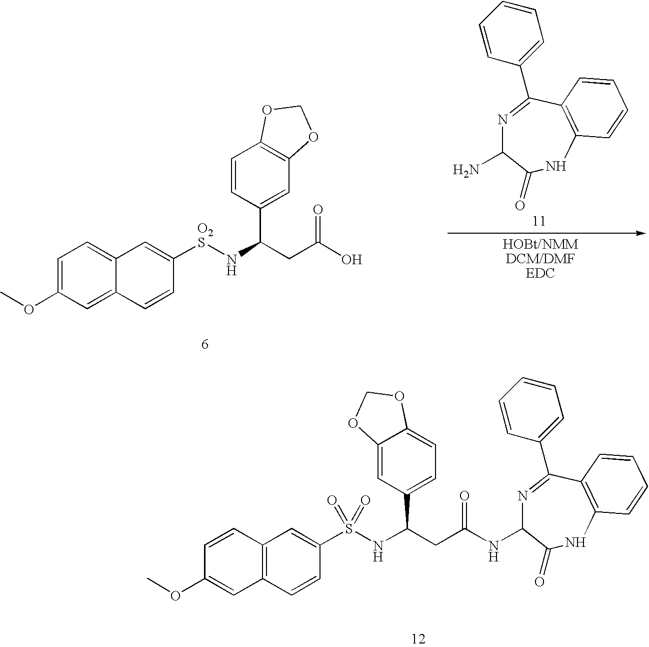

Compound 12 was prepared via the amine 11 (Tyger Scientific, A25300) by a procedure similar to that described for 8 in example 1.

12: 1H-NMR (CD₃OD) δ: 7.98 (s, 1H), 7.56-7.75 (m, 4H), 7.42-7.55 (m, 2H), 7.39 (d, 2H, J=4.4 Hz), 7.14-7.36 (m, 6H), 6.62 (t, 1H, J=8.7 Hz), 6.52 (d, 1H, J=14.5 Hz), 6.43 (dd, 1H, J=7.6 Hz), 5.66 (d, 1H, J=8.6 Hz), 5.48 (d, 1H, J=10.4 Hz), 5.23 (d, 1H, J=15.7 Hz), 4.82 (m, 1H), 3.92 (s, 3H), 2.70-2.92 (m, 2H). 13C-NMR (CDCl₃) δ: 170.84, 170.70, 169.50, 167.65, 167.47, 159.62, 147.28, 146.60, 146.56, 137.61, 136.06, 135.22, 133.62, 133.48, 132.66, 131.19, 131.10, 130.84, 130.10, 129.71, 129.61, 127.94, 127.86, 127.58, 127.05, 126.96, 126.31, 123.47, 122.49, 121.18, 120.21, 120.08, 119.55, 107.00, 106.47, 106.40, 105.18, 100.58, 67.12, 67.04, 55.00, 54.45, 43.12, 43.00, 38.83.

### EXAMPLE 4

### 3-Benzo[1,3]dioxol-5-yl-3-(6-methoxy-naphthalene-2-sulfonylamino)-N-(1-methyl-2-oxo-5-phenyl-2,3-dihydro-1H-benzo[e][1,4]diazepin-3-yl)-propionamide (14)

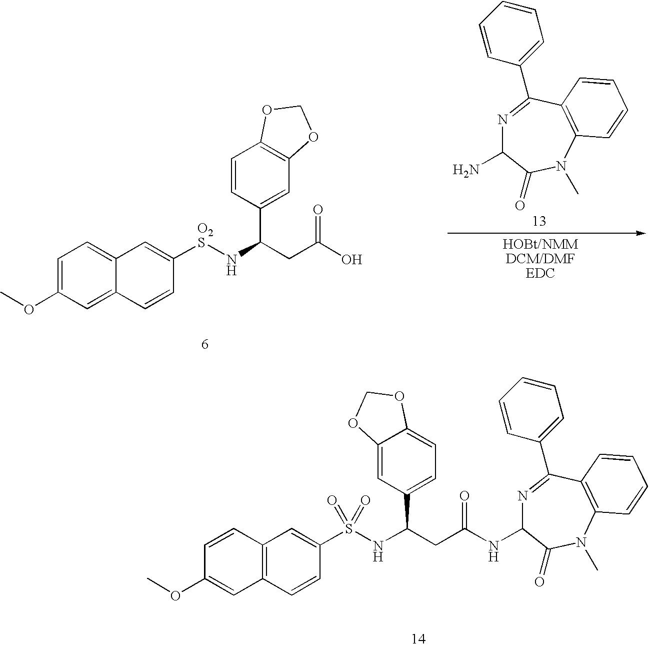

Compound 14 is prepared via the amine 13 (see general procedure 2) by a procedure similar to that described in example 1.

### EXAMPLE 5

### 5-[(2,6-Dichloro-benzenesulfonyl)-methyl-amino]-pentanoic acid[2-(3,4,5,6-tetrahydro-2H-[1,4′]bipyridinyl-4-yl)-ethyl]-amide (20)

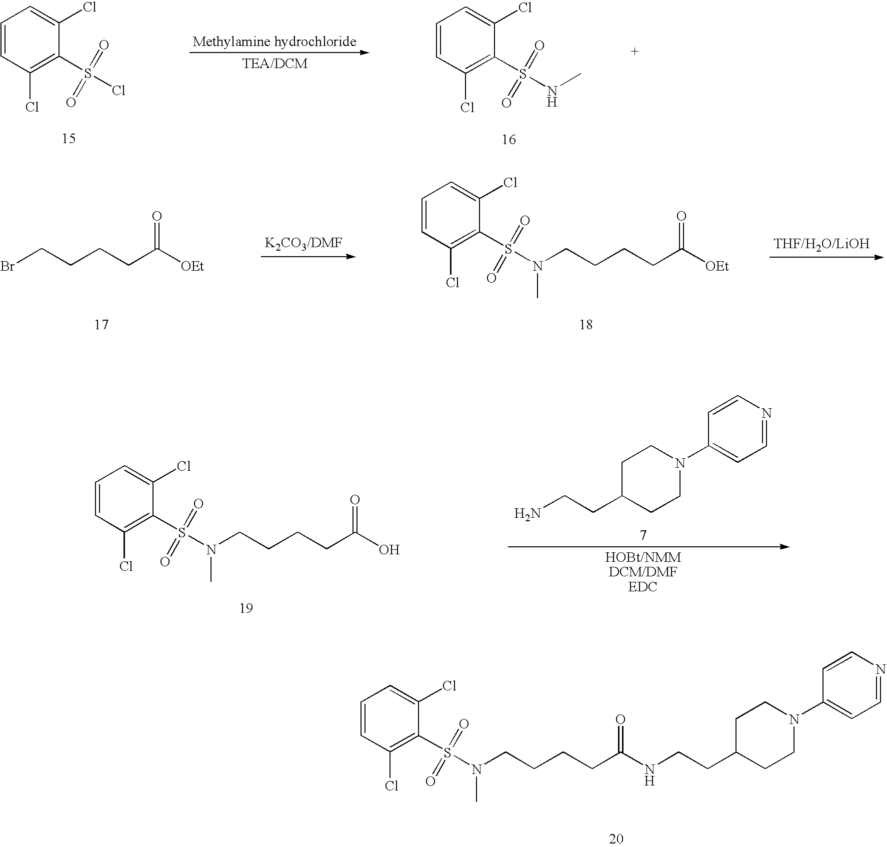

2,6-Dichloro-N-methyl-benzenesulfonamide (16). To a mixture of methylamine hydrochloride (10 mmol, Aldrich, 241016) in dichloromethane (40 mL) is added a solution of 2,6-dichloro-benzenesulfonyl chloride 15 (9 mmol, Aldrich, 545708) in dichloromethane (10 mL). This mixture is stirred for several minutes followed by addition of triethylamine (3.5 mL). The reaction is stirred for 15 hours at room temperature and then quenched with 1M HCl solution (50 mL). The organics are separated and washed with sat. NaHCO₃ solution (50 mL) and dried with sodium sulfate and concentrated. The residue is purified via silica gel chromatography using gradients of hexanes and ethyl acetate.

5-[(2,6-Dichloro-benzenesulfonyl)-methyl-amino]-pentanoic acid ethyl ester (18). To compound 16 (1 mmol) in DMF (10 mL) is added crushed potassium carbonate (3 mmol) and ethyl 5-bromovalerate (1.2 mmol, Aldrich, 129100). The reaction is stirred at room temperature for 15 hours and diluted with ethyl acetate (40 mL) and water (40 mL). The aqueous layer is discarded and the organic layer is washed with water (40 mL) again. The organic layer is separated and dried with sodium sulfate and concentrated. The residue is purified via silica gel chromatography using gradients of hexanes and ethyl acetate.

5-[(2,6-Dichloro-benzenesulfonyl)-methyl-amino]-pentanoic acid (19). Compound 18 (1 mmol) is dissolved in a mixture of THF (2 mL), water (1 mL), and MeOH (1 mL). To this solution is added lithium hydroxide (2 mmol) and the mixture is stirred at room temperature for 3 hours. The organic solvents are removed by rotary evaporation and water (5 mL) is added to the residue. This aqueous mix is then slowly acidified dropwise with conc. HCl until the pH is between 2-3 and a precipitate is formed. The mix is extracted with ethyl acetate (3×10 mL) and the combined organics are washed with water (30 mL) and dried with sodium sulfate and concentrated. The resulting product is used without further purification.

Preparation of the title compound (20). Compound 20 is prepared by a procedure similar to that described for 8 in example 1.

### EXAMPLE 6

5-[(2,6-Dichloro-benzenesulfonyl)-methyl-amino]-pentanoic acid (3,4,5,6-tetrahydro-2H-[1,4′]bipyridinyl-4-ylmethyl)-amide (21). Compound 21 is prepared via amine 9 by a procedure similar to that described for 20 in example 5.

### EXAMPLE 7

5-[(2,6-Dichloro-benzenesulfonyl)-methyl-amino]-pentanoic acid (2-oxo-5-phenyl-2,3-dihydro-1H-benzo[e][1,4]diazepin-3-yl)-amide (22). Compound 22 is prepared via the amine 11 by a procedure similar to that described for 20 in example 5.

### EXAMPLE 8

5-[(2,6-Dichloro-benzenesulfonyl)-methyl-amino]-pentanoic acid (1-methyl-2-oxo-5-phenyl-2,3-dihydro-1H-benzo[e][1,4]diazepin-3-yl)-amide (23). Compound 23 is prepared via the amine 13 by a procedure similar to that described for 20 in example 5.

### EXAMPLE 9

### N-[2-(3,4,5,6-Tetrahydro-2H-[1,4′]bipyridinyl-4-yl)-ethyl]-2-[1-(3-trifluoromethyl-benzenesulfonyl)-piperidin-2-yl]-acetamide (28)

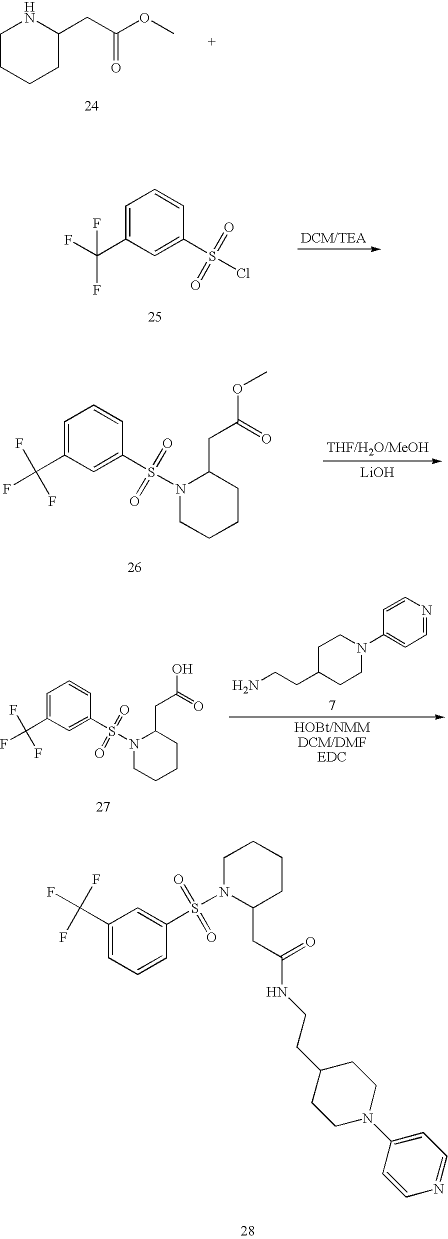

[1-(3-Trifluoromethyl-benzenesulfonyl)-piperidin-2-yl]-acetic acid methyl ester (26). To piperidin-2-yl-acetic acid methyl ester 24 (1 mmol, MicroChemistry Building Blocks, 5483) in dichloromethane (10 mL) is added triethylamine (1 mmol). The reaction is cooled to 0° C. While stirring, 3-trifluoromethyl-benzenesulfonyl chloride 25 (1 mmol, Aldrich, 385417) in dichloromethane (5 mL) is added dropwise under nitrogen. The reaction is allowed to come to room temperature and stirred for 15 hours. The reaction is concentrated and ethyl acetate (30 mL) and water (30 mL) is added. The organic layer is washed and the aqueous layer discarded. The organics are then washed with 1M HCl (20 mL), water (20 mL), sat. NaHCO₃ (20 mL), brine (20 mL) and dried with sodium sulfate and concentrated. The residue is purified via silica gel chromatography using gradients of hexanes and ethyl acetate.

[1-(3-Trifluoromethyl-benzenesulfonyl)-piperidin-2-yl]-acetic acid (27). Compound 27 is prepared by a procedure similar to that described for 19 in example 5.

Preparation of the title compound (28). Compound 28 is prepared by a procedure similar to that described for 8 in example 1.

### EXAMPLE 10

N-(3,4,5,6-Tetrahydro-2H-[1,4′]bipyridinyl-4-ylmethyl)-2-[1-(3-trifluoromethyl-benzenesulfonyl)-piperidin-2-yl]-acetamide (29). Compound 29 is prepared via amine 9 by a procedure similar to that described for 28 in example 9.

### EXAMPLE 11

N-(2-Oxo-5-phenyl-2,3-dihydro-1H-benzo[e][1,4]diazepin-3-yl)-2-[1-(3-trifluoromethyl-benzenesulfonyl)-piperidin-2-yl]-acetamide (30). Compound 30 is prepared via the amine 11 by a procedure similar to that described for 28 in example 9.

### EXAMPLE 12

N-(1-Methyl-2-oxo-5-phenyl-2,3-dihydro-1H-benzo[e][1,4]diazepin-3-yl)-2-[1-(3-trifluoromethyl-benzenesulfonyl)-piperidin-2-yl]-acetamide (31). Compound 31 is prepared via the amine 13 by a procedure similar to that described for 28 in example 9.

### EXAMPLE 13

### N-[2-(3,4,5,6-Tetrahydro-2H-[1,4′]bipyridinyl-4-yl)-ethyl]-2-[(2R)-3-oxo-1-(1,2,3,4-tetrahydro-quinoline-8-sulfonyl)-piperazin-2-yl]-acetamide (39)

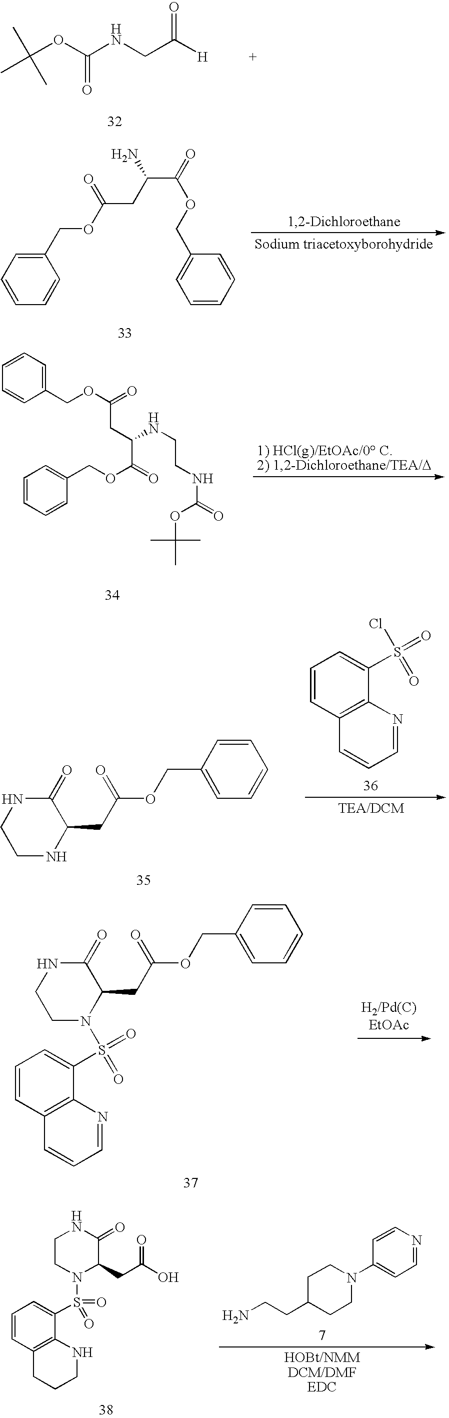

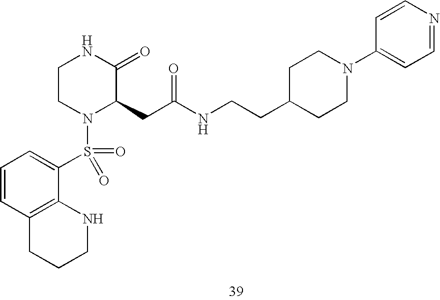

(2R)-2-(2-tert-Butoxycarbonylamino-ethylamino)-succinic acid dibenzyl ester (34). L-aspartic acid dibenzyl ester 33 (10 mmol, Ambinter, 139932922) is dissolved in 1,2-dichloroethane (20 mL). To this solution is added (2-oxo-ethyl)-carbamic acid tert-butyl ester 32 (10 mmol, Aldrich, 472654) and sodium triacetoxyborohydride (15 mmol). The reaction is stirred 15 hours under nitrogen. The reaction mixture is quenched with sat. NaHCO₃ (30 mL) and extracted with dichloromethane (3×20 mL). The combined organic fractions are dried with sodium sulfate and concentrated. The residue is purified via silica gel chromatography using gradients of hexanes and ethyl acetate.

[(2R)-3-Oxo-piperazin-2-yl)]-acetic acid benzyl ester (35). HCl gas is bubbled into a solution of 34 (10 mmol) in EtOAc at 0° C. for 10 minutes. The reaction is concentrated by bubbling air into it for 1 hour. The resulting solid is suspended in 1,2-dichloroethane and triethylamine (30 mmol) is added. The mixture is heated to 90° C. and monitored by HPLC/MS until completed. Upon completion, the reaction mixture is diluted with dichloromethane (100 mL) and washed with water (100 mL), sat. NaHCO₃ (100 mL), brine (100 mL), dried with sodium sulfate and concentrated. The residue is purified via silica gel chromatography using gradients of dichloromethane and methanol with a small amount of ammonium hydroxide as a modifier.

[(2R)-3-oxo-1-(Quinoline-8-sulfonyl)-piperazin-2-yl]-acetic acid benzyl ester (37). Compound 37 is prepared via sulfonyl chloride 36 (Aldrich, 22695) by a procedure similar to that described for 26 in example 9.

[(2R)-3-oxo-1-(1,2,3,4-Tetrahydro-quinoline-8-sulfonyl)-piperazin-2-yl]-acetic acid (38). Compound 37 (100 mmol) is dissolved in EtOAc (40 mL) and placed in Parr shaker bottle. Pd/C (10% by weight, 50 mg) is added. The reaction is shaken on Parr hydrogenation apparatus for 4 hours at 30 PSI. The reaction is filtered through a pad of Celite and concentrated. The crude material is used without further purification.

Preparation of the title compound (39). Compound 39 is prepared by a procedure similar to that described for 8 in example 1.

### EXAMPLE 14

N-(3,4,5,6-Tetrahydro-2H-[1,4′]bipyridinyl-4-ylmethyl)-2-[1-(3-trifluoromethyl-benzenesulfonyl)-piperidin-2-yl]-acetamide (40). Compound 29 is prepared via amine 9 by a procedure similar to that described for 39 in example 13.

### EXAMPLE 15

N-(2-Oxo-5-phenyl-2,3-dihydro-1H-benzo[e][1,4]diazepin-3-yl)-2-[1-(3-trifluoromethyl-benzenesulfonyl)-piperidin-2-yl]-acetamide (41). Compound 30 is prepared via the amine 11 by a procedure similar to that described for 39 in example 13.

### EXAMPLE 16

N-(1-Methyl-2-oxo-5-phenyl-2,3-dihydro-1H-benzo[e][1,4]diazepin-3-yl)-2-[1-(3-trifluoromethyl-benzenesulfonyl)-piperidin-2-yl]-acetamide (42). Compound 31 is prepared via the amine 13 by a procedure similar to that described for 39 in example 13.

### EXAMPLE 17

### Pyrimidine-5-carboxylic acid{1-[2-(3,4,5,6-tetrahydro-2H-[1,4′]bipyridinyl-4-yl)-ethylcarbamoyl]-cyclobutyl}-amide (48)

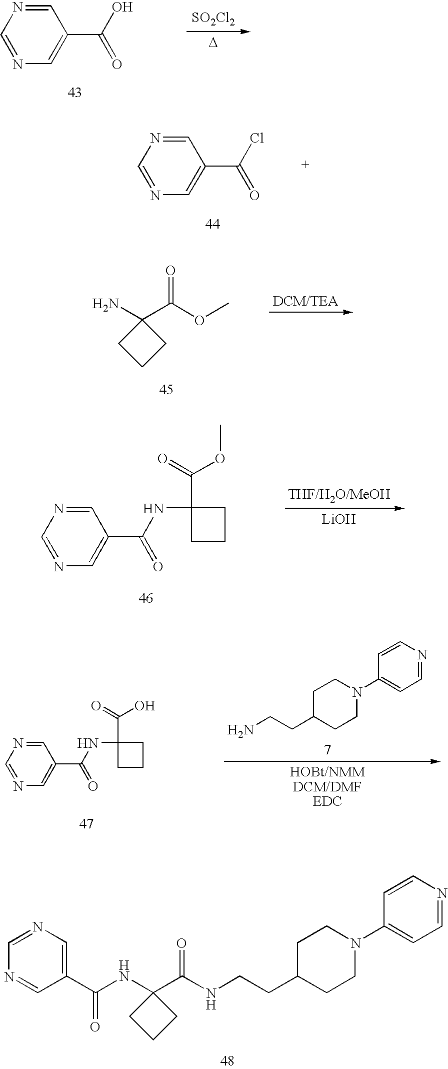

Pyrimidine-5-carbonyl chloride (44). Pyrimidine-5-carboxylic acid 43 (20 mmol, Peakdale Mol. Int., 20044809) and thionyl chloride (20 mL) is refluxed for 2 hours. Excess thionyl chloride is removed via rotary evaporation followed by a protocol of treatment with benzene followed by rotary evaporation. Compound 44 is used without further purification.

1-[(Pyrimidine-5-carbonyl)-amino]-cyclobutanecarboxylic acid methyl ester (46). 1-Amino-cyclobutanecarboxylic acid methyl ester 45 (10 mmol, Aldrich, 630802) is dissolved in dichloromethane (30 mL) and cooled to 0° C. and placed under nitrogen. Compound 44 (10 mmol) in dichloromethane (10 mL) is added drop wise over 10 minutes. The reaction is allowed to warm to room temperature and stirred 15 hours. The mixture is rotovapped to near dryness and taken up in EtOAc (30 mL) and water (30 mL). The aqueous layer is discarded and the organics washed with 1M HCl (30 mL), water (30 mL), sat. NaHCO₃ (30 mL), brine (30 mL) and dried with sodium sulfate and concentrated. The residue is purified via silica gel chromatography using gradients of hexanes and ethyl acetate.

1-[(Pyrimidine-5-carbonyl)-amino]-cyclobutanecarboxylic acid (47). Compound 47 is prepared by a procedure similar to that described for 19 in example 5.

Preparation of the title compound (48). Compound 48 is prepared by a procedure similar to that described for 8 in example 1.

### EXAMPLE 18

Pyrimidine-5-carboxylic acid{1-[(3,4,5,6-tetrahydro-2H-[1,4′]bipyridinyl-4-ylmethyl)-carbamoyl]-cyclobutyl}-amide (49). Compound 49 is prepared via amine 9 by a procedure similar to that described for 48 in example 17.

### EXAMPLE 19

Pyrimidine-5-carboxylic acid[1-(2-oxo-5-phenyl-2,3-dihydro-1H-benzo[e][1,4]diazepin-3-ylcarbamoyl)-cyclobutyl]-amide (50). Compound 50 is prepared via the amine 11 by a procedure similar to that described for 48 in example 17.

### EXAMPLE 20

Pyrimidine-5-carboxylic acid[1-(1-methyl-2-oxo-5-phenyl-2,3-dihydro-1H-benzo[e][1,4]diazepin-3-ylcarbamoyl)-cyclobutyl]-amide (51). Compound 51 is prepared via the amine 13 by a procedure similar to that described for 48 in example 17.

### EXAMPLE 21

### 1-(2-Oxo-5-phenethyl-1-propyl-2,3-dihydro-1H-benzo[e][1,4]diazepin-3-yl)-3-[2-(3,4,5,6-tetrahydro-2H-[1,4′]bipyridinyl-4-yl)-ethyl]-urea (59)

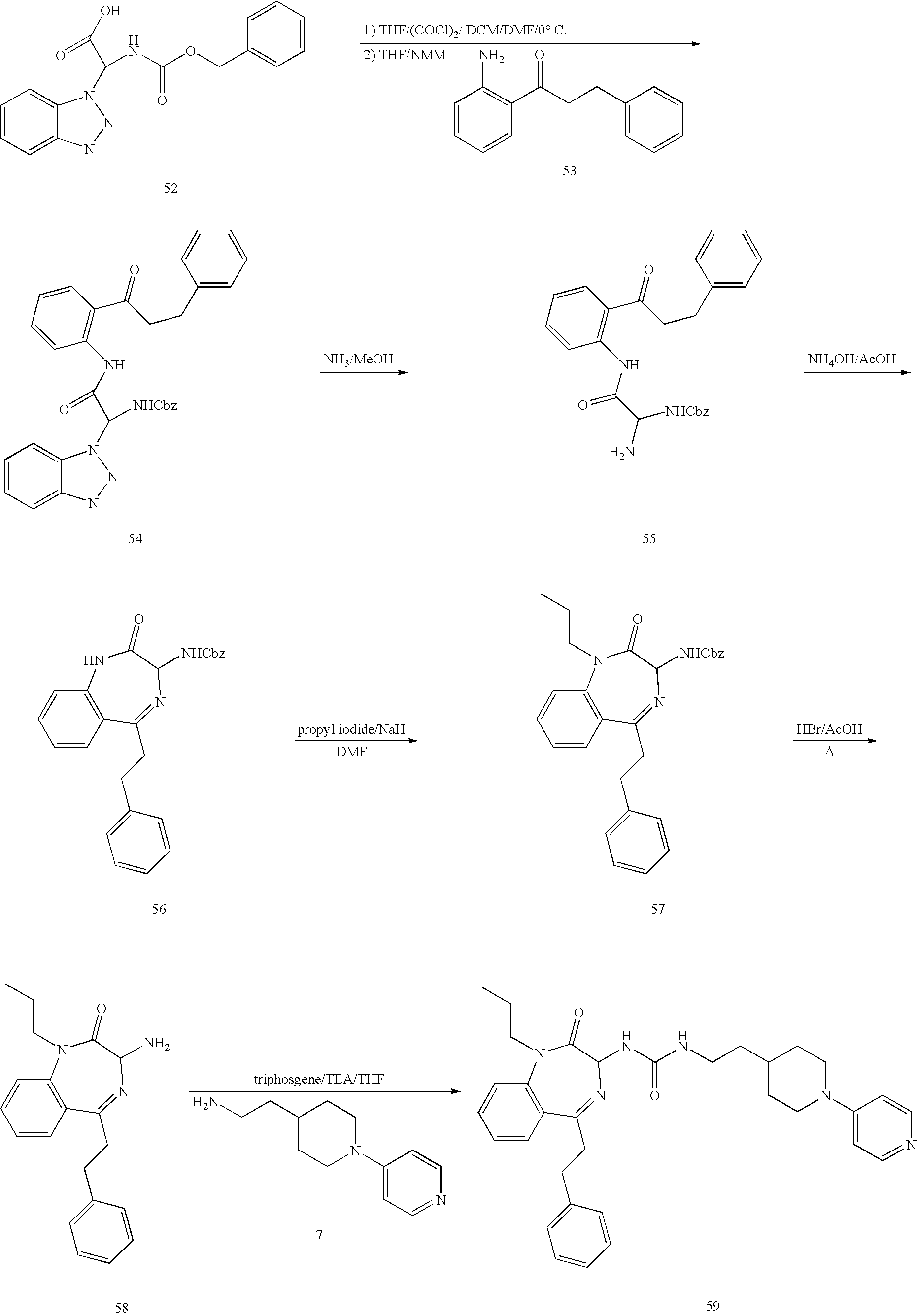

{Benzotriazol-1-yl-[2-(3-phenyl-propionyl)-phenylcarbamoyl]-methyl}-carbamic acid benzyl ester (54). In accordance with J. Org. Chem., 60:730-734 (1995). A solution of benzotriazol-1-yl-benzyloxycarbonylamino-acetic acid 52 (10 mmol, Tiger Scientific, B64161) in anhydrous THF (30 mL) under nitrogen is cooled to 0° C. Oxalyl chloride (10 mmol, 2 N DCM) is added via a syringe followed by DMF (0.2 mL). The reaction is stirred for 2 h at 0° C. A solution of 1-(2-amino-phenyl)-3-phenyl-propan-1-one 53 (9 mmol, Interchim Intermediates, AN-292/40710347) and dry N-methylmorpholine (20 mmol) in anhydrous THF (10 mL) is added dropwise over a 20 min. period. The reaction is warmed to room temp. and filtered. The filter cake is washed with a small amount of THF and the mother liquor is taken on to next reaction without further manipulation.

{Amino-[2-(3-phenyl-propionyl)-phenylcarbamoyl]-methyl}-carbamic acid benzyl ester (55). In accordance with J. Org. Chem., 60:730-734 (1995). The mother liquor from the previous reaction is saturated with ammonia gas, diluted with methanol (50 mL) and saturated once more with ammonia gas for 0.5 h. Ethyl acetate (100 mL) is added and the organics are washed twice with 1 N aqueous sodium hydroxide (20 mL). The remaining organic layer is washed with brine, dried with sodium sulfate, and concentrated in vacuo. No further purification is performed.

(2-Oxo-5-phenethyl-2,3-dihydro-1H-benzo[e][1,4]diazepin-3-yl)-carbamic acid benzyl ester (56). In accordance with J. Org. Chem., 60:730-734 (1995). The crude amine 55 (10 mmol) is dissolved in glacial acetic (50 mL) and combined under nitrogen with ammonium acetate (50 mmol). The reaction is stirred at room temperature for 15 hours. The reaction mixture is concentrated and suspended in ethyl acetate (25 mL) and diethyl ether (75 mL). Aqueous sodium hydroxide (1 N) is added until the pH of the aqueous layer is greater than 8. This mixture is cooled in an ice bath and filtered and the solid is washed with water and diethyl ether and dried under vacuum. No further purification is performed.

(2-Oxo-5-phenethyl-1-propyl-2,3-dihydro-1H-benzo[e][1,4]diazepin-3-yl)-carbamic acid benzyl ester (57). Compound 57 is prepared with propyl iodide by a procedure similar to that for compound (VII) in general procedure 2.

3-Amino-5-phenethyl-1-propyl-1,3-dihydro-benzo[e][1,4]diazepin-2-one (58). A solution of 57 (10 mmol) in glacial acetic acid (100 mL) is saturated with HBr gas. The reaction is warmed to 70° C. and held for 20 minutes. The temperature is then raised to 80° C. and held for another 20 minutes. The mixture is then cooled to room temperature, diluted with diethyl ether (100 mL), sonicated for 30 minutes, and filtered. The resulting hydrobromide salt is free-based via methods known to those skilled in the art. No further purification is performed.

Preparation of the title compound (59). To a stirred solution of triphosgene (1 mmol) in THF (25 mL) is added a solution of 58 (3 mmol) and triethylamine (6 mmol) in dry THF (10 mL) at room temp. The mixture is stirred for 30 min. and a solution of 7 (3 mmol) (see general procedure 1 for preparation) with triethylamine (3 mmol) in THF (10 mL) is added. The mixture is stirred for an additional 30 min. The organics are removed via rotary evaporation and 59 is purified by reverse-phase HPLC.

### EXAMPLE 22

1-(2-Oxo-5-phenethyl-1-propyl-2,3-dihydro-1H-benzo[e][1,4]diazepin-3-yl)-3-(3,4,5,6-tetrahydro-2H-[1,4′]bipyridinyl-4-ylmethyl)-urea (60). Compound 60 is prepared via compound 9 by a procedure similar to that described for 59 in example 21.

### Pharmacetical Formulations

When employed as pharmaceuticals, the compounds of formula (I) are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions that contain, as the active ingredient, one or more of the compounds of formula (I) above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing 5 mg to about 100 mg, more usually about 10 mg to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

The following formulation examples illustrate the pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### FORMULATION EXAMPLE 4

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in water) | |
| Sodium carboxymethyl | 4.5 mg |
| starch | |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50E to 60EC and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

### FORMULATION EXAMPLE 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, cellulose, starch, an magnesium stearate are blended, passed through a No. 20 mesh U.S. sieve, and filled into hard gelatin capsules in 150 mg quantities.

### FORMULATION EXAMPLE 6

Suppositories, each containing 25 mg of active ingredient are made as follows:

| Ingredient | Amount | |
|---|---|---|
| Active Ingredient | 25 | mg |
| Saturated fatty acid | to 2,000 | mg |
| glycerides | | |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### FORMULATION EXAMPLE 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium | 50.0 | mg |
| carboxymethyl | | |
| cellulose (11%) | | |
| Microcrystalline | | |
| cellulose (89%) | | |
| Sucrose | 1.75 | g |
| Sodium benzoate | 10.0 | mg |
| Flavor and Color | q.v. | |
| Purified water | to 5.0 | mL |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 8

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, cellulose, starch, and magnesium stearate are blended, passed through a No. 20 mesh U.S. sieve, and filled into hard gelatin capsules in 560 mg quantities.

### FORMULATION EXAMPLE 9

An intravenous formulation may be prepared as follows:

| Ingredient | Quantity | |
|---|---|---|
| Active Ingredient | 250.0 | mg |
| Isotonic saline | 1000 | mL |

### FORMULATION EXAMPLE 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, which is incorporated herein by reference in its entirety. Such patches may be constructed for continuous, pulsatile, or on demand delivery of pharmaceutical agents.

When it is desirable or necessary to introduce the pharmaceutical composition to the brain, either direct or indirect techniques may be employed. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is incorporated herein by reference in its entirety.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

The following synthetic and biological examples are offered to illustrate this invention and are not to be construed in any way as limiting the scope of this invention. Unless otherwise stated, all temperatures are in degrees Celsius.

### BIOLOGICAL EXAMPLES

The potency and efficacy to inhibit the bradykinin B₁ receptor was determined for the compounds of this invention in a cell-based fluorescent calcium-mobilization assay. The assay measures the ability of test compounds to inhibit bradykinin B₁ receptor agonist-induced increase of intracellular free Ca⁺² in a native human bradykinin B₁ receptor-expressing cell line.

In this example, the following additional abbreviations have the meanings set forth below. Abbreviations heretofore defined are as defined previously. Undefined abbreviations have the art-recognized meanings.
BSA=bovine serum albumin
DMSO=dimethylsulfoxide
FBS=fetal bovine serum
MEM=minimum essential medium
mM=millimolar
ng=nanogram
μg=micrograms
μM=micromolar

Specifically, calcium indicator-loaded cells are pre-incubated in the absence or presence of different concentrations of test compounds followed by stimulation with selective bradykinin B₁ receptor agonist peptide while Ca-dependent fluorescence is monitored.

IMR-90 human lung fibroblast cells (CCL 186, American Type Tissue Collection) are grown in MEM supplemented with 10% FBS as recommended by ATCC. Confluent cells are harvested by trypsinization and seeded into black wall/clear bottom 96-well plates (Costar #3904) at approximately 13,000 cells/well. The following day, cells are treated with 0.35 ng/mL interleukin-1β in 10% FBS/MEM for 2 hours to up-regulate bradykinin B₁ receptors. Induced cells are loaded with fluorescent calcium indicator by incubation with 2.3 μM Fluo-4/AM (Molecular Probes) at 37° C. for 1.5 hrs in the presence of an anion transport inhibitor (2.5 mM probenecid in 1% FBS/MEM). Extracellular dye is removed by washing with assay buffer (2.5 mM probenecid, 0.1% BSA, 20 mM HEPES in Hank's Balanced Salt Solution without bicarbonate or phenol red, pH 7.5) and cell plates are kept in dark until used. Test compounds are assayed at 7 concentrations in triplicate wells. Serial dilutions are made in half log-steps at 100-times final concentration in DMSO and then diluted in assay buffer. Compound addition plates contain 2.5-times final concentrations of test compounds or controls in 2.5% DMSO/assay buffer. Agonist plates contain 5-times the final concentration of 2.5 nM (3×EC50) bradykinin B₁ receptor agonist peptide des-Arg¹⁰-kallidin (DAKD, Bachem) in assay buffer. Addition of test compounds to cell plate, incubation for 5 min at 35° C., followed by the addition of bradykinin B₁ receptor agonist DAKD is carried out in the Fluorometric Imaging Plate Reader (FLIPR, Molecular Devices) while continuously monitoring Ca-dependent fluorescence. Peak height of DAKD-induced fluorescence is plotted as function of concentration of test compounds. IC₅₀ values are calculated by fitting a 4-parameter logistic function to the concentration-response data using non-linear regression (Xlfit, IDBS (ID Business Solutions Ltd.)).

Typical potencies observed for bradykinin B₁ receptor agonist peptides are EC₅₀ approximately 0.8 nM and approximately 100 nM for des-Arg¹⁰-kallidin and des-Arg⁹-bradykinin, respectively, while for bradykinin B₁ receptor antagonist peptide des-Arg¹⁰, Leu⁹-kallidin IC₅₀ is approximately 1 nM.

What is claimed is:

1. A method of preventing or treating conditions which benefit from inhibition of the bradykinin B₁ receptor, comprising:
administering to a host in need thereof a composition comprising a therapeutically effective amount of at least one compound of formula (I), 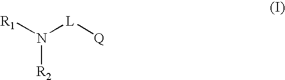
or pharmaceutically acceptable salts thereof, wherein
R₁ is selected from formulae (IIa), (IIb), (IIc), (IId), (IIe), (IIf), and (IIg);
(IIa) is 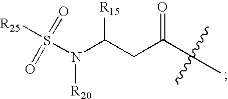
R₁₅, R₂₀, and R₂₅ are independently selected from hydrogen, alkyl, aryl, heteroaryl, alkylaryl, alkylheteroaryl, and —(CH₂)₀₋₆-T;
T is a monocyclic or bicyclic ring system of 5, 6, 7, 8, 9, 10, 11, or 12 atoms, wherein at least one bond in the monocyclic or bicyclic ring system is optionally a double bond, wherein the bicyclic ring system is optionally a fused or spiro ring system, wherein at least one ring in the monocyclic or bicyclic ring system is optionally aromatic, wherein at least one carbon atom in the monocyclic or bicyclic ring system is optionally replaced by a group independently selected from —O—, —C(O)—, —S(O)₀₋₂—, —C(═N—R₆)—, —N—, —NR₆—, —N((CO)₀₋₁R₂₆)—, and —N(SO₂R₂₆)—;
wherein R₁₅, R₂₀, and R₂₅ are independently optionally substituted with at least one R₂₆ group;
R₂₆ is selected from NO₂, CN, halogen, alkyl (optionally substituted with at least one halogen), alkoxy (optionally substituted with at least one halogen), alkylenedioxy (optionally substituted with at least one halogen), benzyloxy, phenyl, —NH₂, —OH, —CF₃, alkylamino, dialkylamino, oxo, —C(O)R₂₇, —COOR₂₇, —C(O)NR₂₇R_{27′}, —NR₂₇C(O)R_{27′}, alkyl, aryl, heteroaryl, cycloalkyl and heterocycloalkyl, each of which is optionally substituted with at least one group independently selected from halogen, —NH₂, —OH, —CN, —CF₃, alkylamino, haloalkyl, oxo, alkoxy, alkoxyalkyl, benzyloxy, alkyl, dialkylamino, —C(O)R₂₇, —COOR₂₇, —C(O)NR₂₇R_{27′}, and NR₂₇C(O)R_{27′};
R₂₇ and R_{27′} are independently selected from H, alkyl, aryl and heteroaryl, each of which is optionally substituted with at least one group independently selected from alkyl, halogen, alkoxy, OH, amino, monoalkylamino, dialkylamino, and CF₃;
(IIb) is 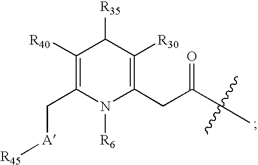
R₃₀ and R₄₀ are independently selected from H, —CO₂H and —CO₂alkyl;
R₃₅ is phenyl optionally substituted with at least one halogen;
A′ is selected from —(CH₂)₀₋₂—, —C(O)—, and —S(O)₀₋₂—;
R₄₅ is selected from azabicycloalkyl, azatricycloalkyl, bicycloalkyl, tricycloalkyl, and phenyl substituted at the 2-position with a group selected from
(a) alkyl optionally substituted with at least one group independently selected from amino, amino-alkoxy, phenylthio, alkyl-phenylthio, dialkylamino-alkoxy, alkylamino-alkoxy, alkylamino, di-alkylamino, hydroxy, alkoxy, piperazinyl, oxopyrrolidinyl, pyrrolidinyl, alkylenedioxy, acyloxy, oxo, morpholino, alkylaminocarbonyl-acylamino, alkoxycarbonyl-acylamino, alkoxycarbonylpiperazinyl, acylpiperazinyl, alkylthio, heterocyclic-alkoxy, (dialkylamino)(cycloalkyl)alkoxy, (alkylamino)(cycloalkyl)alkoxy, (amino)(cycloalkyl)alkoxy, phenylthio, and acylamino;
(b) alkoxy or alkylthio, wherein the alkoxy or alkylthio is optionally substituted with at least one group independently selected from amino, amino-alkoxy, phenylthio, alkyl-phenylthio, di-alkylamino-alkoxy, alkylamino-alkoxy, alkylamino, dialkylamino, hydroxy, alkoxy, piperazinyl, oxopyrrolidinyl, pyrrolidinyl, alkylenedioxy, acyloxy, oxo, morpholino, alkylaminocarbonyl-acylmino, alkoxycarbonyl-acylamino, alkoxycarbonylpiperazinyl, acylpiperazinyl, alkylthio, heterocyclic-alkoxy, (dialkylamino)(cycloalkyl)alkoxy, (alkylamino)(cycloalkyl)alkoxy and (amino)(cycloalkyl)alkoxy;
(c) amino, alkylamino, acylamino, aminoacetylamino, alkylsulfonylamino, halosubstituted-alkylsulfonylamino, halosubstituted-alkylamino and alkoxycarbonylaminoacetylamino;
(d) piperazinylcarbonyl, morpholinocarbony, nitro, cyano, hydroxy, alkylsulfonyl, alkylsulfinyl and di-alkylaminosulphenyl;
(e) alkylthio, acylthio, amino-acylthio, alkylsulfonylthio, halosubstituted-alkylthio and alkoxyaminoacetylthio; and
(f) azacycloalkyl optionally substituted with at least one group independently selected from oxo and alkyl;
(IIc) is 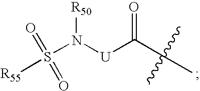
U is selected from alkyl and alkyl-O-alkyl;
R₅₀ is selected from hydrogen and alkyl optionally substituted with at least one group independently selected from halogen, amide, and phenyl;
R₅₅ is selected from hydrogen, alkyl, aryl, alkylaryl, and —(CH₂)₀₋₆-T;
wherein R₅₅ is optionally substituted with at least one R₂₆ group;
(IId) is 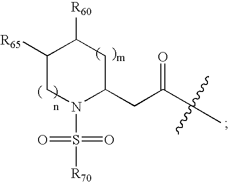
m is 0, 1, or 2;
n is 0, 1, 2, or 3;
R₆₀ and R₆₅ are independently selected from H and alkyl; or
R₆₀ and R₆₅ together form an aryl or heteroaryl ring optionally substituted with at least one group independently selected from halogen, —NH₂, —OH, —CN, —CF₃, alkylamino, oxo, alkoxy, dialkylamino, —C(O)R₂₇, —COOR₂₇, —C(O)NR₂₇R_{27′}, —NR₂₇C(O)R_{27′}, alkyl, aryl, heteroaryl, cycloalkyl and heterocyclyl, each of which is optionally substituted with at least one group independently selected from halogen, —NH₂, —OH, —CN, —CF₃, -alkylamino, haloalkyl, oxo, alkoxy, alkoxyalkyl, alkyl, dialkylamino, —C(O)R₂₇, —COOR₂₇, —C(O)NR₂₇R_{27′}, and NR₂₇C(O)R_{27′};
R₇₀ is selected from hydrogen, alkyl, aryl, alkylaryl, and (CH₂)₀₋₆-T; wherein R₇₀ is optionally substituted with at least one R₂₆ group;
(IIe) is 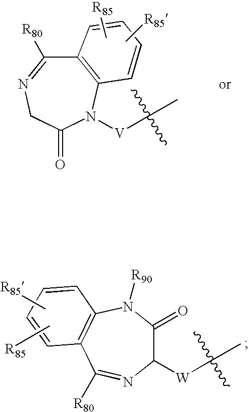
V is —(CH₂)₁₋₇—C(O)—;
W is selected from —NHC(O)—, —NHC(O)—(CH₂)₀₋₄aryl(CH₂)₀₋₄—, NHC(O)—(CH₂)₀₋₄aryl-C(O)—, —NHC(O)NH(CH₂)₀₋₄aryl(CH₂)₀₋₄—, —NHC(O)NH(CH₂)₀₋₄aryl-C(O)—, —OC(O)—, —OC(O)NH(CH₂)₀₋₄aryl(CH₂)₀₋₄—, —OC(O)NH(CH₂)₀₋₄aryl-C(O)—, —(CH₂)₁₋₃—C(O)—, —(CH₂)₁₋₃—C(O)—NH(CH₂)₀₋₄aryl(CH₂)₀₋₄—, and —(CH₂)₁₋₃—C(O)—NH(CH₂)₀₋₄aryl-C(O)—;
R₈₀ is selected from alkyl (optionally substituted with at least one halogen), alkyloxy-, alkyl, -alkyl-cycloalkyl, -alkylaryl, aryl, heteroaryl, and -alkylheteroaryl, wherein the aryl or heteroaryl is optionally substituted with at least one group independently selected from alkyl (optionally substituted with at least one halogen), alkyloxy-, alkylcarboxy-, alkylamido-, OH, halogen, nitro, amino, and cyano, or
R₈₀ is selected from formula (IIIa) and (IIIb), 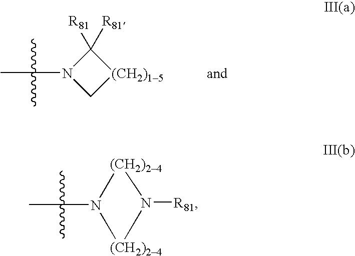
R₈₁ and R_{81′} are independently selected from H, alkyl (optionally substituted with at least one halogen), alkyloxy-, -alkylaryl, aryl, -alkylcycloalkyl, and cycloalkyl;
R₈₅ and R_{85′} are independently selected from H, NO₂, halogen, cyano, OH, amino, alkylthio-, alkyl (optionally substituted with at least one halogen), alkyloxy-, -alkylaryl, aryl, -alkylheteroaryl, heteroaryl, —C(O)—(CH₂)₀₋₂aryl, —C(O)—(CH₂)₀₋₂heteroaryl, —C(O)—O-aryl, —C(O)—O-heteroaryl, —C(O)—NH—(CH₂)₀₋₂aryl, —C(O)—NH(CH₂)₀₋₂heteroaryl, —C(O)—N(alkyl)-(CH₂)₀₋₂aryl, and —C(O)—N(alkyl)(CH₂)₀₋₂heteroaryl, wherein the aryl or heteroaryl is optionally substituted with at least one group independently selected from alkyl, alkyloxy-, alkylcarboxy-, alkylamido-, OH, halogen, nitro, amino, and cyano;
R₉₀ is selected from H, alkyl (optionally substituted with at least one halogen), alkyloxy-, -alkylaryl, -alkyl-cycloalkyl, and cycloalkyl;
(IIf) is 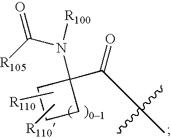
R₁₀₀ and R_{100′} are independently selected from H and alkyl;
R₁₀₅ is selected from alkyl (optionally substituted with at least one group independently selected from hydroxyethyl, halogen, nitro, cyano, —OR₁₁₁, —SR₁₁₁, —COR₁₁₁, —SO₂R₁₁₂, —CO₂R₁₁₁, —OC(O)R₁₁₁, —NR₁₁₃R₁₁₄, —NR₁₁₃C(O)R₁₁₁, —NR₁₁₃CO₂R₁₁₁, —C(O)NR₁₁₃R₁₁₄, and cycloalkyl), cycloalkyl (optionally substituted with at least one group independently selected from halogen, nitro, cyano and phenyl), (CH₂)₀₋₂-aryl (optionally substituted with at least one group independently selected from halogen, nitro, cyano, OR₁₁₁, SR₁₁₁, CO₂R₁₁₁, alkyl and haloalkyl), —(CH₂)₀₋₂-T (optionally substituted with at least one group independently selected from halogen, nitro, cyano, OR₁₁₁, SR₁₁₁, alkyl and haloalkyl), —CO₂R₁₁₁, and —C(O)NR₁₁₃R₁₁₄;
R₁₁₀ and R_{110′} are independently selected from hydrogen, halogen, and alkyl optionally substituted with at least one group independently selected from halogen, OR₁₁₁, OC(O)R₁₁₁, S(O)₀₋₂R₁₁₂, OS(O)₂R₁₁₂, and NR₁₀₀R_{100′}, or
R₁₁₀ and R_{110′} together with the carbon atom to which they are both attached form an exo-cyclic methylene optionally substituted with at least one group selected from alkyl (optionally substituted with at least one halogen) and alkyloxy;
R₁₁₁ is selected from hydrogen, alkyl (optionally substituted with at least one halogen), phenyl (optionally substituted with at least one group independently selected from halogen, cyano, nitro, OH, alkyloxy, cycloalkyl and alkyl optionally substituted with at least one halogen), cycloalkyl, and pyridyl optionally substituted with at least one group independently selected from halogen and alkyl;
R₁₁₂ is selected from alkyl (optionally substituted with at least one halogen), alkyloxy, and phenyl optionally substituted with at least one group independently selected from halogen, cyano, nitro, OH, alkyloxy, cycloalkyl and alkyl optionally substituted with at least one halogen;
R₁₁₃ and R₁₁₄ are independently selected from hydrogen, alkyl (optionally substituted with at least one group independently selected from halogen, amino, monoalkylamino, dialkylamino, and SO₂R₁₁₂), —(CH₂)₀₋₂-phenyl (optionally substituted with at least one group independently selected from halogen, cyano, nitro, OH, alkyloxy, cycloalkyl and alkyl (optionally substituted with at least one halogen)), and cycloalkyl; or
R₁₁₃ and R₁₁₄ together with the nitrogen atom to which they are attached form a 4-, 5-, or 6-membered ring wherein at least one carbon atom within the ring is optionally replaced with a group selected from —N—, —NR₂₆—, —S—, and —O—; or
R₁₁₃ and R₁₁₄ together with the nitrogen atom to which they are attached form a cyclic imide;
(IIg) is 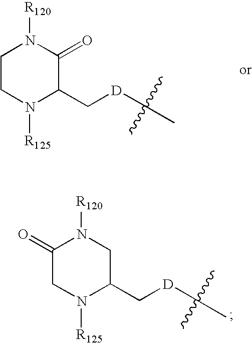
D is selected from —(CH₂)₀₋₆C(O)—, —(CH₂)₀₋₆NR₁₂₁C(O)—, —(CH₂)₀₋₆NR₁₂₁—, —(CH₂)₀₋₆O—, —C(O)—, —(CH₂)₀₋₆CO₂—, —(CH₂)₀₋₆S(O)₀₋₂—, —(CH₂)₀₋₆S—, —HC═CH—, and —(CH₂)₀₋₆—;
R₁₂₀ is selected from hydrogen, alkyl (optionally substituted with at least one halogen), -alkyl-aryl, —S(O)₀₋₂R₁₂₃′, cycloalkyl, —(CH₂)₀₋₆C(O)R₁₂₃, —(CH₂)₀₋₆CO₂R₁₂₃, and —(CH₂)₀₋₆C(O)NR₁₂₁R_{121′};
R₁₂₁ and R_{121′} are independently selected from hydrogen, alkyl (optionally substituted with at least one halogen), cycloalkyl, and aryl optionally substituted with at least one group independently selected from alkyl, halogen, nitro, cyano, OH, —O-alkyl (optionally substituted with at least one halogen), —C(O)OR₁₂₂, —C(O)NR₁₂₂R_{122′}, and —NR₁₂₂R_{122′}; or
R₁₂₁ and R_{121′} and the nitrogen atom to which they are attached together form a 4, 5, 6, or 7 membered ring, optionally; comprising a heteroatom selected from —O—, —S—, and —N(R₆)—;
R₁₂₂ is selected from hydrogen and alkyl;
R₁₂₃ is selected from hydrogen, alkyl (optionally substituted with at least one halogen), cycloalkyl, aryl (optionally substituted with at least one group independently selected from alkyl, halogen, nitro, cyano, —OH, O-alkyl (optionally substituted with at least one halogen), and NR₁₂₁R_{121′}), and heteroaryl optionally substituted with at least one group independently selected from alkyl, halogen, nitro, cyano, —OH, O-alkyl (optionally substituted with at least one halogen), and NR₁₂₁R_{121′};
R_{123′} is selected from alkyl (optionally substituted with at least one halogen), cycloalkyl, aryl (optionally substituted with at least one group independently selected from alkyl, halogen, nitro, cyano, —OH, O-alkyl (optionally substituted with at least one halogen), and NR₁₂₁R_{121′}), and heteroaryl optionally substituted with at least one group independently selected from alkyl, halogen, nitro, cyano, OH, O-alkyl (optionally substituted with at least one halogen), and NR₁₂₁R_{121′};
R₁₂₅ is selected from —(CH₂)₁₋₆CO₂R₁₂₃, —(CH₂)₁₋₆C(O)NR₁₂₁R_{121′}, —S(O)₀₋₂R₁₂₃, —C(O)R₁₂₃, —CO₂R₁₂₃, and alkyl (optionally substituted with at least one group independently selected from halogen, cyano, and aryl (optionally substituted with at least one group independently selected from halogen, cyano, —OR₁₂₃, —NR₁₂₁R_{121′}, —C(O)NR₁₂₁R_{121′}, and phenyl optionally substituted with at least one group independently selected from —CO₂R₁₂₃, halogen, nitro, cyano, —OR₁₂₃, and NR₁₂₁R_{121′}));
R₂ is selected from hydrogen, heterocycloalkyl, heteroaryl, and aryl, wherein the heterocycloalky, heteroaryl, and aryl groups within R₂ are each optionally substituted with at least one R₁₀₅ group;
L is —[C(R₃)(R₄)]₀₋₃—, wherein R₃ and R₄ at each occurrence are independently selected from H and alkyl;
Q is selected from formulae (IVa), (IVb), (IVc), and (IVd);
(IVa) is 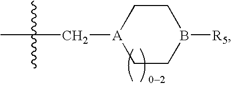
wherein A and B are independently selected from —CH— and —N—;
R₅ is selected from H, heterocycloalkyl, and heteroaryl;
(IVb) is 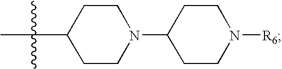
R₆ is selected from H and alkyl;
(IVc) is 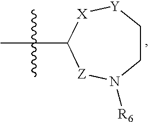
wherein the cyclic ring in formula (IIc) optionally contains at least one double bond;
wherein X is selected from —CH₂—, —N—, and —N(R₇)—;
Y is selected from —NH—, —O—, —S(O)₀₋₂—, N(R₇)(R₈), and —C(R₉)(R₁₀)—;
or X and Y together form a fused aromatic ring; and
(IVd) is 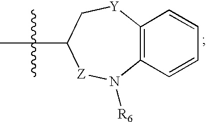
Z is selected from —CH₂— and —C(O)—;
R₇ and R₈ each independently are selected from H and alkyl;
R₉ and R₁₀ each independently are selected from H and phenyl.

2. The method according to claim 1, wherein the formula (I) compound is selected from 3-Benzo[1,3]dioxol-5-yl-3-(6-methoxy-naphthalene-2-sulfonylamino)-N-[2-(3,4,5,6-tetrahydro-2H-[1,4]bipyridinyl-4-yl)-ethyl]-propionamide, 3-Benzo[1,3]dioxol-5-yl-3-(6-methoxy-naphthalene-2-sulfonylamino)-N-(3,4,5,6-tetrahydro-2H-[1,4′]bipyridinyl-4-ylmethyl)-propionamide, 3-Benzo[1,3]dioxol-5-yl-3-(6-methoxy-naphthalene-2-sulfonylamino)-N-(2-oxo-5-phenyl-2,3-dihydro-1H-benzo[e][1,4]diazepin-3-yl)-propionamide, 3-Benzo[1,3]dioxol-5-yl-3-(6-methoxy-naphthalene-2-sulfonylamino)-N-(1-methyl-2-oxo-5-phenyl-2,3-dihydro-1H-benzo[e][1,4]diazepin-3-yl)-propionamide, 5-[(2,6-Dichloro-benzenesulfonyl)-methyl-amino]-pentanoic acid[2-(3,4,5,6-tetrahydro-2H-[1,4′]bipyridinyl-4-yl)-ethyl]-amide, 5-[(2,6-Dichloro-benzenesulfonyl)-methyl-amino]-pentanoic acid (3,4,5,6-tetrahydro-2H-[1,4′]bipyridinyl-4-ylmethyl)-amide, 5-[(2,6-Dichloro-benzenesulfonyl)-methyl-amino]-pentanoic acid (2-oxo-5-phenyl-2,3-dihydro-1H-benzo[e][1,4]diazepin-3-yl)-amide, 5-[(2,6-Dichloro-benzenesulfonyl)-methyl-amino]-pentanoic acid (1-methyl-2-oxo-5-phenyl-2,3-dihydro-1H-benzo[e][1,4]diazepin-3-yl)-amide, N-[2-(3,4,5,6-Tetrahydro-2H-[1,4′]bipyridinyl-4-yl)-ethyl]-2-[1-(3-trifluoromethyl-benzenesulfonyl)-piperidin-2-yl]-acetamide, N-(3,4,5,6-Tetrahydro-2H-[1,4′]bipyridinyl-4-ylmethyl)-2-[1-(3-trifluoromethyl-benzenesulfonyl)-piperidin-2-yl]-acetamide, N-(2-Oxo-5-phenyl-2,3-dihydro-1H-benzo[e][1,4]diazepin-3-yl)-2-[1-(3-trifluoromethyl-benzenesulfonyl)-piperidin-2-yl]-acetamide, N-(1-Methyl-2-oxo-5-phenyl-2,3-dihydro-1H-benzo[e][1,4]diazepin-3-yl)-2-[1-(3-trifluoromethyl-benzenesulfonyl)-piperidin-2-yl]-acetamide, 2-[1-(Naphthalene-2-sulfonyl)-3-oxo-piperazin-2-yl]-N-[2-(3,4,5,6-tetrahydro-2H-[1,4′]bipyridinyl-4-yl)-ethyl]-acetamide, 2-[1-(Naphthalene-2-sulfonyl)-3-oxo-piperazin-2-yl]-N-(3,4,5,6-tetrahydro-2H-[1,4′]bipyridinyl-4-ylmethyl)-acetamide, 2-[1-(Naphthalene-2-sulfonyl)-3-oxo-piperazin-2-yl]-N-(2-oxo-5-phenyl-2,3-dihydro-1H-benzo[e][1,4]diazepin-3-yl)-acetamide, N-(1-Methyl-2-oxo-5-phenyl-2,3-dihydro-1H-benzo[e][1,4]diazepin-3-yl)-2-[1-(naphthalene-2-sulfonyl)-3-oxo-piperazin-2-yl]-acetamide, Pyrimidine-5-carboxylic acid{1-[2-(3,4,5,6-tetrahydro-2H-[1,4′]bipyridinyl-4-yl)-ethylcarbamoyl]-cyclobutyl}-amide, Pyrimidine-5-carboxylic acid{1-[(3,4,5,6-tetrahydro-2H-[1,4′]bipyridinyl-4-ylmethyl)-carbamoyl]-cyclobutyl}-amide, Pyrimidine-5-carboxylic acid[1-(2-oxo-5-phenyl-2,3-dihydro-1H-benzo[e][1,4]diazepin-3-ylcarbamoyl)-cyclobutyl]-amide, Pyrimidine-5-carboxylic acid[1-(1-methyl-2-oxo-5-phenyl-2,3-dihydro-1H-benzo[e][1,4]diazepin-3-ylcarbamoyl)-cyclobutyl]-amide, 1-(2-Oxo-5-phenethyl-1-propyl-2,3-dihydro-1H-benzo[e][1,4]diazepin-3-yl)-3-[2-(3,4,5,6-tetrahydro-2H-[1,4′]bipyridinyl-4-yl)-ethyl]-urea, and 1-(2-Oxo-5-phenethyl-1-propyl-2,3-dihydro-1H-benzo[e][1,4]diazepin-3-yl)-3-(3,4,5,6-tetrahydro-2H-[1,4′]bipyridinyl-4-ylmethyl)-urea.

3. The method according to claim 1 wherein R₂ is hydrogen.

4. The method according to claim 1 wherein R₁ is selected from (R)-4-(naphthalen-2-ylsulfonyl)-3-(2-oxopropyl)piperazin-2-one, (R)-N-(1-(benzo[d][1,3]dioxol-5-yl)-3-oxobutyl)-6-methoxynaphthalene-2-sulfonamide, 1-(1-(3-(trifluoromethyl)phenylsulfonyl)piperidin-2-yl)propan-2-one, 2,6-dichloro-N-methyl-N-(5-oxohexyl)benzenesulfonamide, N-(1-acetylcyclobutyl)pyrimidine-5-carboxamide, and (Z)-N-(2-oxo-5-phenethyl-1-propyl-2,3-dihydro-1H-benzo[e][1,4]diazepin-3-yl)formamide.

5. The method according to claim 1, wherein Q is selected from (Z)-1,3-dimethyl-5-phenyl-1H-benzo[e][1,4]diazepin-2(3H)-one, (Z)-1-methyl-5-phenyl-1H-benzo[e][1,4]diazepin-2(3H)-one, (Z)-5-phenyl-1H-benzo[e][1,4]diazepin-2(3H)-one, 4-(4-ethylpiperidin-1-yl)pyridine, and 4-(4-methylpiperidin-1-yl)pyridine.

6. A selective antagonist of bradykinin B₁ receptor over bradykinin B₂ receptor wherein said selective antagonist of bradykinin B₁ receptor is a compound of formula (I): 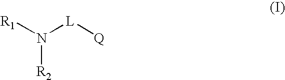
or pharmaceutically acceptable salts, prodrugs or isomers thereof, wherein R₁, R₂, L, and Q are as defined as in claim 1.

7. A method for selectively inhibiting bradykinin B₁ receptor over bradykinin B₂ receptor by administering to a host in need thereof an effective amount of at least one compound of formula (I), 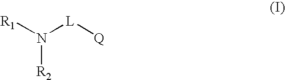
or a stereoisomer, or pharmaceutically acceptable salt thereof; wherein R₁, R₂, L, and Q are defined as in claim 1.

8. A pharmaceutical composition comprising, a pharmaceutically acceptable carrier and a therapeutically effective amount of at least one compound of formula (I), 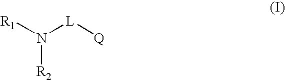
or mixtures thereof, effective to treat or ameliorate adverse symptoms in mammals mediated by bradykinin B₁ receptor; wherein R₁, R₂, L, and Q are defined as in claim 1.

9. A method for treating or ameliorating adverse symptoms in mammals mediated by bradykinin B₁ receptor comprising, administering a therapeutically effective amount of at least one compound of formula (I), 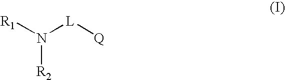
or mixtures thereof, wherein R₁, R₂, L, and Q are defined as in claim 1.

10. A pharmaceutical composition comprising a pharmaceutically acceptable carrier and a therapeutically effective amount of at least one compound of formula (I), 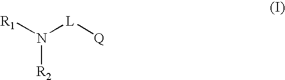
or mixtures thereof, effective to treat or ameliorate adverse symptoms in mammals associated with up-regulating bradykinin B₁ receptor following tissue damage or inflammation, wherein R₁, R₂, L, and Q are as defined as in claim 1.

11. A method for treating or ameliorating adverse symptoms in mammals associated with up-regulating bradykinin B₁ receptor following tissue damage or inflammation comprising, administering a therapeutically effective amount of at least one compound of formula (I), 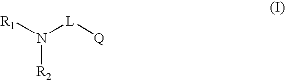
or mixtures thereof, or a stereoisomer, or pharmaceutically acceptable salt thereof, wherein R₁, R₂, L, and Q are as defined as in claim 1.

12. A method for treating or ameliorating adverse symptoms associated with the presence or secretion of bradykinin B₁ receptor agonists in mammals comprising, administering a therapeutically effective amount of at least one compound of formula (I), 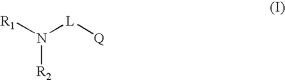
or mixtures thereof, wherein R₁, R₂, L, and Q are as defined as in claim 1.

13. A method for treating or ameliorating pain, inflammation, septic shock, or the scarring process in mammals mediated by bradykinin B₁ receptor comprising, administering a therapeutically effective amount of at least one compound of formula (I), 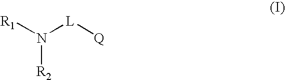
or mixtures thereof, wherein R₁, R₂, L, and Q are as defined as in claim 1.

14. A method for treating or ameliorating adverse symptoms associated with up-regulating bradykinin B₁ receptor relative to burns, perioperative pain, migraine, shock, central nervous system injury, asthma, rhinitis, premature labor, inflammatory arthritis, inflammatory bowel disease, or neuropathic pain comprising, administering a therapeutically effective amount of at least one compound of formula (I), 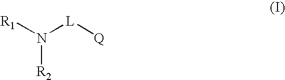
or mixtures thereof, wherein R₁, R₂, L, and Q are as defined as in claim 1.

15. A method for treating or ameliorating adverse symptoms associated with the presence or secretion of bradykinin B₁ receptor agonists in mammals comprising, administering a therapeutically effective amount of at least one compound of formula (I), 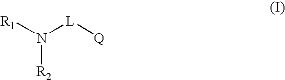
or mixtures thereof, wherein R₁, R₂, L, and Q are as defined as in claim 1.

16. A method for determining bradykinin B₁ receptor agonist levels in a biological sample comprising, contacting said biological sample with at least one compound of formula (I), 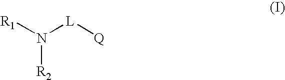
at a predetermined concentration, wherein R₁, R₂, L, and Q are as defined as in claim 1.

17. A packaged pharmaceutical composition for treating conditions which benefit from inhibition of the bradykinin B₁ receptor, comprising:
(a) a container which holds an effective amount of at least one compound of formula (I) or a pharmaceutically acceptable salt thereof, 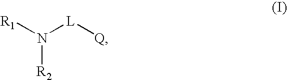
wherein R₁, R₂, L, and Q are as defined as in claim 1; and
(b) instructions for using the pharmaceutical composition.

18. A pharmaceutical composition for treating conditions which benefit from inhibition of the bradykinin B₁ receptor, comprising: a therapeutically effective amount of at least one compound of formula (I), 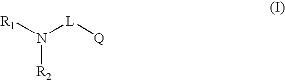
or pharmaceutically acceptable salts thereof, wherein R₁, R₂, L, and Q are as defined as in claim 1.

19. An article of manufacture comprising:
(a) a medicament comprising: an effective amount of at least one compound of formula (I), 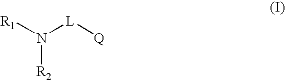
in combination with active and/or inactive pharmaceutical agents, wherein R₁, R₂, L, and Q are as defined as in claim 1;
(b) a package insert providing that an effective amount of at least one compound of formula (I) should be administered to a patient in need of therapy for disorders, conditions or diseases which benefit from inhibition of the bradykinin B₁ receptor; and
(c) a container in which a medicament comprising: an effective amount of at least one compound of formula (I) in combination with active and/or inactive pharmaceutical agents is stored.


SEQUENCE LISTING (6 sequences)

SEQ ID NO: 1 - protein, 9 aa, Artificial Sequence
  Description of Artificial Sequence Synthetic peptide
RPPGFSPFR

SEQ ID NO: 2 - protein, 10 aa, Artificial Sequence
  Description of Artificial Sequence Synthetic peptide
KRPPGFSPFR

SEQ ID NO: 3 - protein, 11 aa, Artificial Sequence
  Description of Artificial Sequence Synthetic peptide
MKRPPGFSPFR

SEQ ID NO: 4 - protein, 11 aa, Artificial Sequence
  Description of Artificial Sequence Synthetic peptide
ISRPPGFSPFR

SEQ ID NO: 5 - protein, 9 aa, Artificial Sequence
  Description of Artificial Sequence Synthetic peptide
RPXGFSPFR

SEQ ID NO: 6 - protein, 9 aa, Artificial Sequence
  Description of Artificial Sequence Synthetic peptide
RPPXFSPFR